SEQUENCE LISTING (46 sequences)

SEQ ID NO: 1 - protein, 10 aa, Artificial Sequence
  Synthetic Construct
LALGETTRPA

SEQ ID NO: 2 - protein, 10 aa, Artificial Sequence
  Synthetic Construct
LANETITRPA

SEQ ID NO: 3 - protein, 10 aa, Artificial Sequence
  Synthetic Construct
LAKAGQANNA

SEQ ID NO: 4 - protein, 10 aa, Artificial Sequence
  Synthetic Construct
LAKDPKTTNA

SEQ ID NO: 5 - protein, 7 aa, Artificial Sequence
  Synthetic Construct
KDTDTTR

SEQ ID NO: 6 - protein, 7 aa, Artificial Sequence
  Synthetic Construct
RAGGSVG

SEQ ID NO: 7 - protein, 7 aa, Artificial Sequence
  Synthetic Construct
AVDTTKF

SEQ ID NO: 8 - protein, 7 aa, Artificial Sequence
  Synthetic Construct
STGKVPN

SEQ ID NO: 9 - protein, 10 aa, Artificial Sequence
  Synthetic Construct
LAKDTDTTRA

SEQ ID NO: 10 - protein, 10 aa, Artificial Sequence
  Synthetic Construct
LARAGGSVGA

SEQ ID NO: 11 - protein, 10 aa, Artificial Sequence
  Synthetic Construct
LAAVDTTKFA

SEQ ID NO: 12 - protein, 10 aa, Artificial Sequence
  Synthetic Construct
LASTGKVPNA

SEQ ID NO: 13 - protein, 735 aa, Artificial Sequence [sequence omitted]

SEQ ID NO: 14 - protein, 7 aa, Artificial Sequence
  Synthetic Construct
LGETTRP

SEQ ID NO: 15 - protein, 7 aa, Artificial Sequence
  Synthetic Construct
NETITRP

SEQ ID NO: 16 - protein, 7 aa, Artificial Sequence
  Synthetic Construct
KAGQANN

SEQ ID NO: 17 - protein, 7 aa, Artificial Sequence
  Synthetic Construct
KDPKTTN

SEQ ID NO: 18 - protein, 7 aa, Artificial Sequence
  Synthetic Construct
KDTDTTR

SEQ ID NO: 19 - protein, 7 aa, Artificial Sequence
  Synthetic Construct
RAGGSVG

SEQ ID NO: 20 - protein, 7 aa, Artificial Sequence
  Synthetic Construct
AVDTTKF

SEQ ID NO: 21 - protein, 7 aa, Artificial Sequence
  Synthetic Construct
STGKVPN

SEQ ID NO: 22 - DNA, 293 bp, Artificial Sequence
  Synthetic Construct
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattacca

SEQ ID NO: 23 - DNA, 220 bp, Artificial Sequence
  Synthetic Construct
tgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccg

SEQ ID NO: 24 - DNA, 318 bp, Artificial Sequence
  Synthetic Construct
ctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcga

SEQ ID NO: 25 - DNA, 102 bp, Artificial Sequence
  Synthetic Construct
ccagctgttggggtgagtactccctctcaaaagcgggcattacttctgcgctaagattgtcagtttccaaaaacgaggaggatttgatattcacctggcccg

SEQ ID NO: 26 - DNA, 810 bp, Artificial Sequence
  Synthetic Construct
ctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagtta

SEQ ID NO: 27 - DNA, 202 bp, Artificial Sequence
  Synthetic Construct
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 28 - DNA, 68 bp, Artificial Sequence
  Synthetic Construct
aggcggtcttgagacggcggatggtcgaggtgaggtgtggcaggcttgagatccagctgttggggtga

SEQ ID NO: 29 - DNA, 129 bp, Artificial Sequence
  Synthetic Construct
cgctgttttgacctccatagtggacaccgggaccgatccagcctccgcgtctcaggggagatctcgtttagtgaaccgtcagatcctcactctcttccgcatcgctgtctgcgagggccagctgttggg

SEQ ID NO: 30 - DNA, 36 bp, Artificial Sequence
  Synthetic Construct
ttgatattcacctggcccgatctggccatacacttg

SEQ ID NO: 31 - DNA, 22 bp, Artificial Sequence
  Synthetic Construct
cccaggtccaagtttaaacgcc

SEQ ID NO: 32 - DNA, 77 bp, Artificial Sequence
  Synthetic Construct
tctttgggctttctatatacattttgaaatgaggttgacaagttacctaggaaaactgtcttcctgcccgggtggca

SEQ ID NO: 33 - DNA, 1172 bp, Artificial Sequence
  Synthetic Construct
ctctggagacgacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagaggccatccacgctgttttgacctccatagtggacaccgggaccgatccagcctccgcgtctcaggggagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccaccatg

SEQ ID NO: 34 - DNA, 1043 bp, Artificial Sequence
  Synthetic Construct
actgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttacctaggaaaactgtcttcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgaagacagtcag

SEQ ID NO: 35 - protein, 431 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - DNA, 1377 bp, Artificial Sequence
  Synthetic Construct
atggtcagctactgggacaccggggtcctgctgtgcgcgctgctcagctgtctgcttctcacaggatctagttccggaagtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 37 - protein, 7 aa, Artificial Sequence
  Synthetic Construct
HKFKSGD

SEQ ID NO: 38 - protein, 10 aa, Artificial Sequence
  Synthetic Construct
  VARIANT 1, 2, 10: Xaa = Any Amino Acid
XXHKFKSGDX

SEQ ID NO: 39 - protein, 10 aa, Artificial Sequence
  Synthetic Construct
LAHKFKSGDA

SEQ ID NO: 40 - protein, 724 aa, Artificial Sequence
  Synthetic Construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 41 - protein, 725 aa, Artificial Sequence
  Synthetic Construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPTTGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 42 - protein, 724 aa, Artificial Sequence
  Synthetic Construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMLAHKFKSGDAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 43 - protein, 725 aa, Artificial Sequence
  Synthetic Construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMLAHKFKSGDAPTTGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 44 - protein, 587 aa, Artificial Sequence
  Synthetic Construct
APTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMLAHKFKSGDAPTTGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 45 - protein, 532 aa, Artificial Sequence
  Synthetic Construct
MSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMLAHKFKSGDAPTTGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 46 - protein, 745 aa, Artificial Sequence
  Synthetic Construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

(19) United States
(12) Patent Application Publication
(10) Pub. No.: US 2020/0297869 A1
(43) Pub. Date: Sep. 24, 2020

(54) SEQUENTIAL INTRAVITREAL ADMINISTRATION OF AAV GENE THERAPY TO CONTRALATERAL EYES

(71) Applicant: Adverum Biotechnologies, Inc., Redwood City, CA (US)

(72) Inventors: Diana CEPEDA, Mountain View, CA (US); Mehdi GASMI, San Diego, CA (US)

(21) Appl. No.: 16/808,932

(22) Filed: Mar. 4, 2020

Related U.S. Application Data
(60) Provisional application No. 62/839,457, filed on Apr. 26, 2019.
(60) Provisional application No. 62/813,597, filed on Mar. 4, 2019.

(51) Int. Cl. A61K 48/00; C12N 15/86; C07K 16/22; A61P 27/02

(52) U.S. Cl. CPC A61K 48/005; C12N 15/86; A61K 9/0019; C07K 16/22; A61P 27/02; A61K 48/0075

(57) ABSTRACT

Provided are methods of treating an ocular disease or disorder in a subject, comprising: administering a unit dose of a pharmaceutical composition to a first eye the subject via intravitreal (IVT) injection at a first time point, and administering a second unit dose of the pharmaceutical composition to a contralateral eye of the subject via IVT injection at a second time point.

### CROSS REFERENCE TO RELATED APPLICATIONS

This application claims the priority benefit of U.S. Provisional Application No. 62/813,597, filed Mar. 4, 2019; and U.S. Provisional Application No. 62/839,457, filed Apr. 26, 2019; the contents of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 627002001000SEQLIST.TXT, date recorded: Mar. 3, 2020, size: 62 KB).

### FIELD

The present disclosure relates to methods of treating ocular diseases and disorders in a subject that comprise administering a recombinant adeno associated virus (rAAV) that comprises a heterologous nucleic acid sequence encoding, e.g., aflibercept to a first eye of the subject via intravitreal (IVT) injection at a first time point, and administering the rAAV to a contralateral eye of the subject via IVT injection at a second time point.

### BACKGROUND

Gene delivery vectors based on adeno-associated viruses (AAV) have demonstrated promise for the treatment of a variety of ocular diseases and disorders. However, administration of AAV-based ocular therapies remains a challenge. For example, subretinally injected AAV can efficiently transduce retinal pigment epithelium and photoreceptors in primate retina, but this mode of delivery entails puncturing the retina, which may lead to retinal tears, retinal detachment, and loss of vision. While intravitreal (IVT) AAV injection is less invasive than subretinal injection, intravitreally delivered viral vector capsid is not confined to the immune-privileged retinal compartment. Exposure of AAV capsid epitopes to the adaptive immune system in the vitreous fluid leads to the development of neutralizing antibodies (“nAb”), which may prevent effective vector re-administration. Certain ocular diseases may develop asynchronously in each eye of a patient. These asynchronous ocular diseases include wet age-related macular degeneration (wAMD), retinal vein occlusion (RVO), diabetic eye disease (DED), diabetic macular edema (DME), diabetic retinopathy (DR), choroidal neovascularization (CNV), and retinopathy of prematurity. There is, therefore, a need in the art for safe and effective methods for treating ocular diseases in each of a subject's eyes at sequential time points.

### SUMMARY OF THE DISCLOSURE

Provided herein is a method of treating an ocular disease or disorder in a subject, comprising: (i) administering a first unit dose of a pharmaceutical composition to a first eye of the subject via intravitreal (IVT) injection at a first time point, and (ii) administering a second unit dose of the pharmaceutical composition to a contralateral eye of the subject via IVT injection at a second time point, wherein the pharmaceutical composition comprises: (a) a recombinant adeno-associated virus (rAAV) particle comprising a nucleic acid encoding an anti-vascular endothelial growth factor (VEGF) agent, wherein the rAAV particle is capable of infecting a retinal cell following IVT injection and (b) a pharmaceutically acceptable excipient. In some embodiments the method further comprises a step of measuring a level of neutralizing antibodies against the rAAV in a sample from the subject following the first time point and prior to the second time point. In some embodiments, the method further comprises a step of measuring expression level of the nucleic acid encoding the anti-vascular endothelial growth factor (VEGF) agent in a sample from the subject following the first time point and prior to the second time point. In some embodiments, the time interval between the first time point and the second time point is at least about 2 weeks. In some embodiments, the time interval between the first time point and the second time point at least about 4 weeks or about 1 month. In some embodiments, the time interval between the first time point and the second time point is at least about 6 weeks. In some embodiments, the time interval between the first time point and the second time point is at least about 8 weeks or about 2 months. In some embodiments, the first unit dose and the second unit dose each comprise between about 1E9 and about 3E13 vector genomes.

In some embodiments, the first unit dose and the second unit dose each comprise between about 1E10 and about 1E13 vector genomes (i.e., per eye). In some embodiments, the first unit dose and the second unit dose each comprise between about 1E11 and about 1E13 vector genomes (i.e., per eye). In some embodiments, the first unit dose and the second unit dose each comprise between about 2E11 and about 6E11 vector genomes (i.e., between about 2E11 and about 6E11 vector genomes per eye). In some embodiments, the first unit dose and the second unit dose each comprise between about 2E11 and about 6E12 vector genomes (i.e., between about 2E11 and about 6E12 vector genomes per eye). In some embodiments, the second unit dose is higher than the first unit dose. In some embodiments, the second unit dose is about 300% of the first unit dose (such as 3-fold or 3 times the first unit dose). In some embodiments, the second unit dose is between about 300% and about 1000% of the first unit dose. In some embodiments, the first unit dose comprises about 6E10 vector genomes the second unit dose comprises between about 1.8E11 and about 6E11 vector genomes. In some embodiments, the first unit dose comprises about 6E11 vector genomes and the second unit dose comprises between about 1.8E12 and about 6E12 vector genomes. In some embodiments, wherein the first unit dose comprises about 2E11 vector genomes and the second unit dose comprises between about 6E11 and 2E12 about vector genomes. In some embodiments, the first unit dose comprises about 2E12 vector genomes and the second unit dose comprises between about 6E12 and about 2E13 vector genomes. In some embodiments, the volumes of first unit dose and the second unit dose are each no more than about 100 μL. In some embodiments, the volumes of first unit dose and the second unit dose are each no more than about 50 μL.

Also provided herein is a method treating an ocular disease or disorder in a subject, comprising: administering a unit dose of a pharmaceutical composition to one eye of the subject via intravitreal (IVT) injection, wherein the pharmaceutical composition comprises: (a) a recombinant adeno-associated virus (rAAV) particle comprising a nucleic acid encoding an anti-vascular endothelial growth factor (VEGF) agent, wherein the rAAV particle is capable of infecting a retinal cell following IVT injection, and (b) a pharmaceutically acceptable excipient, and wherein the subject was administered with a prior unit dose of the pharmaceutical composition to a contralateral eye via IVT injection. In some embodiments, the method further comprises a step of measuring a level of neutralizing antibodies against the rAAV in a sample from the subject following administration of the prior unit dose to the contralateral eye and prior to the administration of the unit dose the one eye. In some embodiments, the method further comprises a step of measuring expression level of the nucleic acid encoding the anti-vascular endothelial growth factor (VEGF) agent in a sample from the subject following administration of the prior unit dose to the contralateral eye and prior to the administration of the unit dose to the one eye. In some embodiments, the unit dose comprises between about 1E10 and about 1E13 vector genomes. In some embodiments, the unit dose comprises between about 2E11 and about 6E11 vector genomes. In some embodiments, the unit dose comprises between about 2E12 and about 6E12 vector genomes. In some embodiments, the prior unit dose comprised between about 1E10 and about 1E13 vector genomes. In some embodiments, the prior unit dose comprised between about 2E11 and about 6E11 vector genomes. In some embodiments, the prior unit dose comprised between about 2E12 and about 6E12 vector genomes. In some embodiments, the unit dose administered to the one eye is higher than the prior unit dose administered to the contralateral eye. In some embodiments, the unit dose is at least about 300% of the prior unit dose. In some embodiments, the unit dose is between about 300% and about 1000% of the prior unit dose. In some embodiments, the prior unit dose comprised about 6E10 vector genomes and the unit dose comprises between about 1.8E11 and about 6E11 vector genomes. In some embodiments, the prior unit dose comprised about 6E11 vector genomes and the unit dose comprises between about 1.8E12 and about 6E12 vector genomes. In some embodiments, the prior unit dose comprised about 2E11 vector genomes and the unit dose comprises between about 6E11 and 2E12 about vector genomes. In some embodiments, the prior unit dose comprised about 2E12 vector genomes and the unit dose comprises between about 6E12 and about 2E13 vector genomes. In some embodiments, the time interval between the administration of the prior unit dose and administration of the unit dose is at least about 2 weeks. In some embodiments, the time interval between the administration of the prior unit dose and administration of the unit dose is at least about 4 weeks or about 1 month. In some embodiments, the time interval between the administration of the prior unit dose and administration of the unit dose is at least about 6 weeks. In some embodiments, the time interval between the administration of the prior unit dose and administration of the unit dose is at least about 8 weeks or about two months.

In some embodiments, the rAAV particle comprises a variant capsid protein that comprises a peptide insertion relative to a corresponding parental AAV capsid protein, wherein the peptide insertion has an amino acid sequence selected from LALGETTRPA (SEQ ID NO: 1); LANETITRPA (SEQ ID NO: 2), LAKAGQANNA (SEQ ID NO: 3), LAKDPKTTNA (SEQ ID NO: 4), KDTDTTR (SEQ ID NO: 5), RAGGSVG (SEQ ID NO: 6), AVDTTKF (SEQ ID NO: 7), STGKVPN (SEQ ID NO: 8), LAKDTDTTRA (SEQ ID NO: 9), LARAGGSVGA (SEQ ID NO: 10), LAAVDTTKFA (SEQ ID NO: 11), and LASTGKVPNA (SEQ ID NO: 12), wherein the insertion site is located between two adjacent amino acids at a position between amino acids corresponding to amino acids 570 and 611 of VP1 of AAV2 or the corresponding position in the capsid protein of another AAV serotype. In some embodiments, the rAAV particle is an rAAV2 particle that comprises a variant capsid protein comprising the amino acid sequence LALGETTRPA (SEQ ID NO: 1) inserted between positions 587 and 588 of SEQ ID NO: 13. In some embodiments, the variant capsid protein comprises the amino acid sequence of SEQ ID NO: 46.

In some embodiments, the rAAV particle comprises a variant capsid protein that comprises a modified sequence, the modified sequence comprising one or more amino acid substitutions within amino acid residues 570-579 relative to a parental AAV capsid protein, wherein the modified sequence comprises HKFKSGD (SEQ ID NO: 37), and wherein the amino acid residue numbering corresponds to an AAV5 VP1 capsid protein. In some embodiments, the parental AAV capsid protein is an AAV5 capsid protein or an AAV5 and AAV2 hybrid capsid protein. In some embodiments, the parental AAV capsid protein is a AAV2.5T capsid protein. In some embodiments, the parental AAV capsid protein is an AAV2.5T VP1 capsid protein. In some embodiments, the modified sequence comprises LAHKFKSGDA (SEQ ID NO: 39). In some embodiments, the variant AAV capsid protein comprises a capsid sequence having at least 85% homology to the amino acid sequence set forth in SEQ ID NO: 40 or SEQ ID NO: 41. In some embodiments, the variant AAV capsid protein comprises a capsid sequence set forth in SEQ ID NO: 42 or SEQ ID NO:43.

In some embodiments, the anti-VEGF agent is a bevacizumab, brolucizumab, or ranibizumab. In some embodiments, the anti-VEGF agent is a polypeptide that comprises an amino acid sequence having at least 80% homology to aflibercept. In some embodiments, the anti-VEGF agent is aflibercept. In some embodiments, the retinal cell is a photoreceptor, a retinal ganglion cell, a Müller cell, a bipolar cell, an amacrine cell, a horizontal cell, or a retinal pigmented epithelium cell. In some embodiments, the ocular disease or disorder is choroidal neovascularization, wet age-related macular degeneration (wAMD), macular edema following retinal vein occlusion, diabetic macular edema (DME), or diabetic retinopathy associated with DME. In some embodiments, the ocular disease or disorder is choroidal neovascularization or wet AMD. In some embodiments, the subject is a human. In some embodiments, the subject is responsive to administration of an anti-VEGF agent, wherein the anti-VEGF agent is a polypeptide. In some embodiments, the anti-VEGF agent is aflibercept. In some embodiments, the subject received prior treatment for the ocular disease or disorder with an anti-VEGF agent. In some embodiments, the anti-VEGF agent was aflibercept.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### INCORPORATION BY REFERENCE

All references cited herein, including patent applications and publications, are hereby incorporated by reference in their entirety.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows a time course of average aflibercept expression in the vitreous and aqueous fluids of the right eyes of three African green monkeys that were administered IVT with AAV2.7m8-aflibercept to the right eye on Day 0, and to the left eye on Day 59.

FIG. 1B shows a time course of average aflibercept expression in the vitreous and aqueous fluids of the left eyes of three African green monkeys that were administered IVT with AAV2.7m8-aflibercept to the right eye on Day 0, and to the left eye on Day 59.

FIG. 2A shows a time course of aflibercept expression in the vitreous fluid of the right eyes of each of the three African green monkeys that were administered IVT with AAV2.7m8-aflibercept to the right eye on Day 0, and to the left eye on Day 59.

FIG. 2B shows a time course of aflibercept expression in the aqueous fluid of the right eyes of each of the three African green monkeys that were administered IVT with AAV2.7m8-aflibercept to the right eye on Day 0, and to the left eye on Day 59.

FIG. 3A shows a time course of aflibercept expression in the vitreous fluid of the left eyes of each of the three African green monkeys that were administered IVT with AAV2.7m8-aflibercept to the right eye on Day 0, and to the left eye on Day 59.

FIG. 3B shows a time course of aflibercept expression in the aqueous fluid of the left eyes of each of the three African green monkeys that were administered IVT with AAV2.7m8-aflibercept to the right eye on Day 0, and to the left eye on Day 59.

FIG. 4 shows aflibercept expression levels at study termination (i.e., on Day 264) in various tissues of the right eyes and left eyes of each African green monkey that was administered IVT with AAV2.7m8-aflibercept to the right eye on Day 0 and to the left eye on Day 59.

FIG. 5A shows a time course of neutralizing antibody (nAb) response to 7m8 capsid protein in the vitreous liquid of the left eyes and right eyes of the three African green monkeys that were administered IVT with AAV2.7m8-aflibercept to the right eye on Day 0 and to the left eye on Day 59.

FIG. 5B shows a time course of neutralizing antibody (nAb) response to 7m8 capsid protein in the sera of the three African green monkeys that were administered IVT with AAV2.7m8-aflibercept to the right eye on Day 0 and to the left eye on Day 59.

FIG. 6 provides the nucleic acid sequence of aflibercept (SEQ ID NO: 36).

FIG. 7 provides graphs summarizing the results of monthly assessments of the levels of inflammatory keratic precipitate, vitreous cell infiltrates, aqueous haze, and aqueous cell infiltrates in the right eyes (i.e., first eyes) and left eyes (i.e., later dosed eyes) of monkeys who were administered IVT with AAV2.7m8-aflibercept as compared to a monkey that received IVT vehicle injection. Arrows with dotted lines indicate time of injection of AAV2.7m8-aflibercept.

FIG. 8 provides the results of optical coherence tomography measurements taken monthly to determine the effects of staggered bilateral dosing of AAV2.7m8-aflibercept on retinal thickness and retinal volume.

FIG. 9 provides sections of retinal tissue that were stained with haematoxylin and eosin in order to assess retinal morphology, including cell death and immune cell infiltration.

### DETAILED DESCRIPTION OF THE DISCLOSURE

The methods, compositions, and kits described herein may employ, unless otherwise indicated, conventional techniques and descriptions of molecular biology (including recombinant techniques), cell biology, biochemistry, immunochemistry and ophthalmic techniques, which are within the skill of those who practice in the art. Such conventional techniques include methods for observing and analyzing the retina, or vision in a subject, cloning and propagation of recombinant virus, formulation of a pharmaceutical composition, and biochemical purification and immunochemistry. Specific illustrations of suitable techniques can be had by reference to the examples herein. However, equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, et al., Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Stryer, L., Biochemistry (4th Ed.) W.H. Freeman, N.Y. (1995); Gait, “Oligonucleotide Synthesis: A Practical Approach” IRL Press, London (1984); Nelson and Cox, Lehninger, Principles of Biochemistry, 3rd Ed., W.H. Freeman Pub., New York (2000); and Berg et al., Biochemistry, 5th Ed., W.H. Freeman Pub., New York (2002), all of which are herein incorporated by reference in their entirety for all purposes.

### Definitions

Unless otherwise defined, all technical terms used herein have the same meaning as commonly understood by one of ordinary skill in the art.

The terminology used herein is for the purpose of describing particular examples only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The term “comprising” as used herein is synonymous with “including” or “containing”, and is inclusive or open-ended.

Any reference to “or” herein is intended to encompass “and/or” unless otherwise stated. As used herein, the term “about” a number refers to that number plus or minus 10% of that number. The term “about” a range refers to that range minus 10% of its lowest value and plus 10% of its greatest value.

The term “subject”, “patient”, or “individual” refers to primates, such as humans and non-human primates, e.g., African green monkeys and rhesus monkeys. In some embodiments, the subject is a human.

The terms “treat,” “treating”, “treatment,” “ameliorate” or “ameliorating” and other grammatical equivalents as used herein, refer to alleviating, abating or ameliorating an ocular disease or disorder or symptoms of the ocular disease or disorder, preventing additional symptoms of the ocular disease or disorder, ameliorating or preventing the underlying metabolic causes of symptoms, inhibiting the ocular disease or disorder, e.g., arresting the development of the ocular disease or disorder, relieving the ocular disease or disorder, causing regression of the ocular disease or disorder, or stopping the symptoms of the ocular disease or disorder, and are intended to include prophylaxis. The terms further include achieving a therapeutic benefit and/or a prophylactic benefit. The term “therapeutic benefit” refers to eradication or amelioration of the ocular disease or disorder being treated. Also, a therapeutic benefit is achieved with the eradication or amelioration of one or more of the physiological symptoms associated with the ocular disease or disorder such that an improvement is observed in the patient, notwithstanding that, in some embodiments, the patient is still afflicted with the ocular disease or disorder. For prophylactic benefit, the pharmaceutical compositions are administered to a patient at risk of developing the ocular disease or disorder, or to a patient reporting one or more of the physiological symptoms of the ocular disease or disorder, even if a diagnosis of the disease or disorder has not been made. Patients with asynchronous disease development may receive therapeutic benefit from treatment of their eye with more advanced disease, and prophylactic benefit from treatment of their eye with less advanced disease.

The terms “administer,” “administering”, “administration,” and the like, as used herein, can refer to the methods that are used to enable delivery of therapeutics or pharmaceutical compositions to the desired site of biological action. These methods include intravitreal or subretinal injection to an eye.

The terms “effective amount”, “therapeutically effective amount” or “pharmaceutically effective amount” as used herein, can refer to a sufficient amount of at least one pharmaceutical composition or compound being administered which will relieve to some extent one or more of the symptoms of the ocular disease or disorder being treated. An “effective amount”, “therapeutically effective amount” or “pharmaceutically effective amount” of a pharmaceutical composition may be administered to a subject in need therein if as a unit dose (as described in further detail elsewhere herein).

The term “pharmaceutically acceptable” as used herein, can refer to a material, such as a carrier or diluent, which does not abrogate the biological activity or properties of a compound disclosed herein, and is relatively nontoxic (i.e., when the material is administered to an individual it does not cause undesirable biological effects nor does it interact in a deleterious manner with any of the components of the composition in which it is contained).

The term “pharmaceutical composition,” or simply “composition” as used herein, can refer to a biologically active compound, optionally mixed with at least one pharmaceutically acceptable chemical component, such as, though not limited to carriers, stabilizers, diluents, dispersing agents, suspending agents, thickening agents, excipients and the like.

An “AAV vector” or “rAAV vector” as used herein refers to an adeno-associated virus (AAV) vector or a recombinant AAV (rAAV) vector comprising a polynucleotide sequence not of AAV origin (e.g., a polynucleotide heterologous to AAV such as a nucleic acid sequence that encodes a therapeutic transgene, e.g., aflibercept) for transduction into a target cell or to a target tissue. In general, the heterologous polynucleotide is flanked by at least one, and generally by two, AAV inverted terminal repeat sequences (ITRs). The term rAAV vector encompasses both rAAV vector particles and rAAV vector plasmids. A rAAV vector may either be single-stranded (ssAAV) or self-complementary (scAAV).

An “AAV virus” or “AAV viral particle” or “rAAV vector particle” or “rAAV particle” refers to a viral particle comprising at least one AAV capsid protein (typically by all of the capsid proteins of a wild-type AAV) and a polynucleotide rAAV vector. If the particle comprises a heterologous polynucleotide (e.g., a polynucleotide other than a wild-type AAV genome such as a transgene to be delivered to a target cell or target tissue), it is typically referred to as an “rAAV vector particle” or an “rAAV vector”. Thus, production of rAAV particle necessarily includes production of an rAAV vector, as such a vector contained within an rAAV particle.

The term “packaging” as used herein can refer to a series of intracellular events that can result in the assembly and encapsidation of a rAAV particle.

AAV “rep” and “cap” genes refer to polynucleotide sequences encoding replication and encapsidation proteins of adeno-associated virus. AAV rep and cap are referred to herein as AAV “packaging genes.”

The term “polypeptide” can encompass both naturally-occurring and non-naturally occurring proteins (e.g., a fusion protein), peptides, fragments, mutants, derivatives and analogs thereof. A polypeptide may be monomeric, dimeric, trimeric, or polymeric. Further, a polypeptide may comprise a number of different domains each of which has one or more distinct activities. For the avoidance of doubt, a “polypeptide” may be any length greater two amino acids.

As used herein, “polypeptide variant” or simply “variant” refers to a polypeptide whose sequence contains an amino acid modification. In some embodiments, the modification is an insertion, duplication, deletion, rearrangement or substitution of one or more amino acids compared to the amino acid sequence of a reference protein or polypeptide, such as a native or wild-type protein. A variant may have one or more amino acid point substitutions, in which a single amino acid at a position has been changed to another amino acid, one or more insertions and/or deletions, in which one or more amino acids are inserted or deleted, respectively, in the sequence of the reference protein, and/or truncations of the amino acid sequence at either or both the amino or carboxy termini A variant can have the same or a different biological activity compared to the reference protein, or the unmodified protein.

In some embodiments, a variant can have, for example, at least about any one of 80%, 85%, 90%, 91%, 92%, 93%, 94%, 95%, 96%, 97%, 98%, 99% overall sequence homology to its counterpart reference protein. In some embodiments, a variant can have at least about 90% overall sequence homology to the wild-type protein. In some embodiments, a variant exhibits at least about 95%, at least about 98%, at least about 99%, at least about 99.5%, or at least about 99.9% overall sequence identity.

As used herein, “recombinant” can refer to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. Thus, for example, a protein synthesized by a microorganism is recombinant, for example, if it is synthesized from an mRNA synthesized from a recombinant gene present in the cell.

The term “anti-VEGF agent” includes any therapeutic agent, including proteins, polypeptides, peptides, fusion protein, multimeric proteins, gene products, antibody, human monoclonal antibody, antibody fragment, aptamer, small molecule, kinase inhibitor, receptor or receptor fragment, or nucleic acid molecule, that can reduce, interfere with, disrupt, block and/or inhibit the activity or function of an endogenous VEGF and/or an endogenous VEGF receptor (VEGFR), or the VEGF-VEGFR interaction or pathway in vivo. An anti-VEGF agent can be any one of the known therapeutic agents that can reduce new blood vessel growth or formation and/or edema, or swelling, when delivered into a cell, tissue, or a subject in vivo, e.g., ranibizumab, brolucizumab, or bevacizumab. In some embodiments, an anti-VEGF agent can be naturally occurring, non-naturally occurring, or synthetic. In some embodiments, an anti-VEGF agent can be derived from a naturally occurring molecule that was subsequently modified or mutated to confer an anti-VEGF activity. In some embodiments, an anti-VEGF agent is a fusion or chimeric protein. In such proteins, functional domains or polypeptides are artificially fused to a moiety or a polypeptide to make a fusion or chimeric protein that can sequester VEGF in vivo or function as a VEGFR decoy. In some embodiments, an anti-VEGF agent is a fusion or chimeric protein that blocks endogenous VEGFR from interacting with its ligands.

As used herein, “VEGF” can refer to any isoform of VEGF, unless required otherwise, including, but not limited to, VEGF-A, VEGF-B, VEGF-C, VEGF-D, VEGF-E, VEGF-F, or any combination, or any functional fragment or variant thereof. Unless required otherwise, “VEGF” can refer to any member of the VEGF family, including members: VEGF-A, placenta growth factor (PGF), VEGF-B, VEGF-C, and VEGF-D, or any combination, functional fragment, or variant thereof. As used herein, “VEGF receptor” or “VEGFR” or “VEGF-R” can be used to refer to any one of the receptors of VEGF, including, but not limited to, VEGFR-1 (or Flt-1), VEGFR-2 (or Flk-1/KDR), and VEGFR-3 (or Flt-4). VEGFR can be a membrane bound or soluble form, or a functional fragment or truncation of a receptor. Examples of anti-VEGF agent include, but are not limited to, ranibizumab, bevacizumab, brolucizumab, or any combination, variant, or functional fragment thereof.

“Operatively linked” or “operably linked” or “coupled” can refer to a juxtaposition of genetic elements, wherein the elements are in a relationship permitting them to operate in an expected manner. For instance, a promoter can be operatively linked to a coding region if the promoter helps initiate transcription of the coding sequence. There may be intervening residues between the promoter and coding region so long as this functional relationship is maintained.+++

The term “expression vector” or “expression construct” or “cassette” or “plasmid” or simply “vector” can include any type of genetic construct, including AAV or rAAV vectors, containing a nucleic acid or polynucleotide coding for a gene product in which part or all of the nucleic acid encoding sequence is capable of being transcribed and is adapted for gene therapy. The transcript can be translated into a protein. In some embodiments, the transcript is partially translated or not translated. In certain aspects, expression includes both transcription of a gene and translation of mRNA into a gene product. In other aspects, expression only includes transcription of the nucleic acid encoding genes of interest. An expression vector can also comprise control elements operatively linked to the encoding region to facilitate expression of the protein in target cells. The combination of control elements and a gene or genes to which they are operably linked for expression can sometimes be referred to as an “expression cassette,” a large number of which are known and available in the art or can be readily constructed from components that are available in the art.

The term “heterologous” can refer to an entity that is genotypically distinct from that of the rest of the entity to which it is being compared. For example, a polynucleotide introduced by genetic engineering techniques into a plasmid or vector derived from a different species can be a heterologous polynucleotide. A promoter removed from its native coding sequence and operatively linked to a coding sequence with which it is not naturally found linked can be a heterologous promoter.

As used herein, “7m8” refers to the amino acid sequence LALGETTRPA (SEQ ID NO: 1).

“7m8 variant” refers to a rAAV, which can be of any serotype, with the amino acid sequence LALGETTRPA (SEQ ID NO: 1) inserted in the solvent exposed GH loop of the capsid protein.

When 7m8 is inserted in a rAAV2 (also referred to as AAV2.7m8), the amino acid sequence LALGETTRPA (SEQ ID NO: 1) is inserted into the GH loop of the AAV2 capsid protein, e.g., between positions 587 and 588 of the AAV2 capsid protein, VP1. When 7m8 is inserted in a rAAV1 (also referred to as AAV1.7m8), the amino acid sequence LALGETTRPA (SEQ ID NO: 1) is inserted into the GH loop of the AAV1 capsid protein, e.g., between amino acids 590 and 591 of the AAV1 capsid protein. When 7m8 is inserted in a rAAV5 (also referred to as AAV5.7m8), the amino acid sequence LALGETTRPA (SEQ ID NO: 1) is inserted into the GH loop of the AAV5 capsid protein, e.g., between amino acids 575 and 576 of the AAV5 capsid protein. When 7m8 is inserted in a rAAV6 (also referred to as AAV6.7m8), the amino acid sequence LALGETTRPA (SEQ ID NO: 1) is inserted into the GH loop of the AAV6 capsid protein, e.g., between amino acids 590 and 591 of the AAV6 capsid protein. When 7m8 is inserted in a rAAV7 (also referred to as AAV7.7m8), the amino acid sequence LALGETTRPA (SEQ ID NO: 1) is inserted into the GH loop of the AAV7 capsid protein, e.g., between amino acids 589 and 590 of the AAV7 capsid protein. When 7m8 is inserted in a rAAV8 (also referred to as AAV8.7m8), the amino acid sequence LALGETTRPA (SEQ ID NO: 1) is inserted into the GH loop of the AAV8 capsid protein, e.g., between amino acids 590 and 591 of the AAV8 capsid protein. When 7m8 is inserted in a rAAV9 (also referred to as AAV9.7m8), the amino acid sequence LALGETTRPA (SEQ ID NO: 1) is inserted into the GH loop of the AAV9 capsid protein, e.g., between amino acids 588 and 589 of the AAV9 capsid protein. When 7m8 is inserted in a rAAV10 (also referred to as AAV10.7m8), the amino acid sequence LALGETTRPA (SEQ ID NO: 1) is inserted into the GH loop of the AAV10 capsid protein, e.g., between amino acids 589 and 590 of the AAV10 capsid protein.

### Overview

The ability of AAV vectors (e.g., AAV2.7m8) to efficiently transduce target retinal cells following IVT injection has been exploited to successfully transfer therapeutic genes into photoreceptors, retinal pigment epithelium, and the inner retina to treat a variety of retinal diseases. Intravitreal (IVT) AAV administration is a safe and convenient method of retinal delivery, but it has been suggested that neutralizing antibodies (nAb) against the vector capsid are more likely to be generated following IVT injection than following subretinal injection. Given that certain ocular diseases, such as wet age-related macular degeneration (wAMD), can develop asynchronously in both of an individual's eyes, there is a concern that nAb generated following IVT administration of an AAV to a first eye may decrease the efficiency of therapeutic gene transfer and prevent effective vector re-administration, e.g., to the individual's contralateral eye. The methods provided herein are based on Applicant's finding that that development of immunity (e.g., neutralizing antibodies or “nAb”) following IVT administration of an rAAV2-based vector to a subject's first eye does not completely block transduction following administration of the vector via IVT injection to the subject's contralateral eye.

### Methods of Treatment

Provided herein is a method of treating, slowing the progression of, and/or preventing an ocular disease or disorder in a subject that comprises (i) administering a first unit dose of a pharmaceutical composition to a first eye of the subject via intravitreal (IVT) injection at a first time point, and (ii) administering a second unit dose of the pharmaceutical composition to a contralateral eye of the subject via IVT injection at a second time point, wherein the pharmaceutical composition comprises: (a) a recombinant adeno-associated virus (rAAV) particle comprising a nucleic acid encoding an anti-vascular endothelial growth factor (VEGF) agent, wherein the rAAV particle is capable of infecting a retinal cell following IVT injection and (b) a pharmaceutically acceptable excipient.

In some embodiments, the method further comprises a step of measuring a level of neutralizing antibodies (nAbs) against the rAAV in a sample from the subject following the first time point and prior to the second time point. In some embodiments, the sample is a serum sample, a vitreous fluid sample, or an aqueous fluid sample. In some embodiments, measuring the level of nAbs comprises measuring the potency of neutralizing antibodies (nAbs) against the rAAV. In some embodiments, the neutralizing potency is quantified by the inhibitory concentration (IC), defined as the concentration of nAb (e.g., serum nAb) at which rAAV infectivity has been reduced by 50% relative to the absence of nAb (i.e., IC50). In some embodiments, rAAV particle infectivity is measured in an in vitro cell-based assay in a cell type that is readily transduced by the rAAV, for example HEK293T cells. In some embodiments IC50 is expressed as the dilution factor necessary for a nAb-containing sample to cause a 50% reduction in rAAV particle infectivity. For example, where a nAb-containing sample required a 1:500 dilution to cause a 50% reduction in rAAV infectivity, the IC50 may be expressed as 500. In some embodiments, the IC50 is less than about any one of 600, 550, 500, 450, 400, 350, 300, 250, 200, 125, or 100. In some embodiments, the time interval between the first time point and the second time point is at least about any one of 1, 2, 3, 4, 5, 6, 7, 8, 9, or 10 weeks, at least about any one of 1, 2, 3, 4, 5, 6, 7, 8, 9, 10, 11, or 12 months, or at least about any one of 0.5, 1, 1.5, 2, 2.5, 3, 3.5, 4, 4.5, 5, 5.5, 6, 6.5, 7, 7.5, 8, 8.5, 9, 10, 15, or 20 years, including any range between these values, and the step of measuring the level of neutralizing antibodies (nAbs) against the rAAV in the sample from the subject occurs within the time interval.

In some embodiments, the step of measuring the levels of nAbs against the rAAV in a sample from the subject following the first time point and prior to the second time point is accomplished by means of a cut-point nAb assay. In a cut-point assay, the level of inhibition of rAAV infectivity caused by a test sample is compared to a predetermined cut point, above which the sample is determined to be positive for nAbs, and below which the sample is determined to be negative. The cut point is set at the level below which a predetermined percentage (e.g. 95%) of test samples taken from an rAAV-naïve population fall.

In some embodiments, the method further comprises a step of measuring expression level of the nucleic acid encoding the anti-VEGF agent in a sample from the subject following the first time point and prior to the second time point. In some embodiments, the sample is a vitreous fluid sample or an aqueous fluid sample. In some embodiments, the expression level of the nucleic acid is measured by determining the abundance (e.g., relative abundance) of mRNA encoding the anti-VEGF agent. In some embodiments, the abundance (e.g., relative abundance) of mRNA is determined (e.g., quantified) via northern blot, RT-qPCR, RNA sequencing, RNA in situ hybridization, or other methods known in the art. In some embodiments, the expression level of the nucleic acid is measured by determining the abundance (e.g., relative abundance) of the anti-VEGF agent encoded by the nucleic acid. In some embodiments, the abundance (e.g., relative abundance) of anti-VEGF agent is determined (e.g., quantified) via western blot, Liquid chromatography-mass spectrometry (LC/MS), ELISA, immunohistochemistry, or other immunoassays known in the art. In some embodiments, the time interval between the first time point and the second time point is at least about any one of 1, 2, 3, 4, 5, 6, 7, 8, 9, or 10 weeks; at least about any one of 1, 2, 3, 4, 5, 6, 7, 8, 9, 10, 11, or 12 months; or at least about any one of 0.5, 1, 1.5, 2, 2.5, 3, 3.5, 4, 4.5, 5, 5.5, 6, 6.5, 7, 7.5, 8, 8.5, 9, 10, 15, or 20 years, including any range between these values, and the step of measuring the expression level of the nucleic acid encoding the anti-VEGF agent in a sample from the subject occurs within the time interval.

In some embodiments the first and second unit doses are each therapeutically effective doses, e.g., doses sufficient to ensure efficient delivery of the nucleic acid (e.g., the nucleic acid encoding an anti-VEGF agent, such as aflibercept) into target cells (such as retinal) cells. In some embodiments, the first unit dose and the second unit dose are the same, e.g., between about 1E9 to about 3E13 vector genomes, between about 1E10 and about 1E13 vector genomes, between about 1E11 and 1E13 vector genomes, between about 1E10 to about 3E12 vector genomes, or between about 2E12 and about 6E12 vector genomes. In some embodiments, the second unit dose is higher than the first unit dose, e.g., at least about any one of 150%, 175%, 200%, 225%, 250%, 275%, or 300%, 350%, 400%, 450%, 500%, 550%, 600%, 650% 700%, 750%, 800%, 850%, 900%, 950%, or 1000% of the first unit dose, including any range in between these values. For example, in some embodiments, the second unit dose is at least about any one of 1.5-fold, 1.75-fold, 2-fold, 2.25-fold, 2.5-fold, 2.75-fold, 3-fold, 3.25-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, or 10-fold of the first unit dose, including any range in between these values. In some embodiments, the first unit dose comprises about 6E10 vector genomes and the second unit dose comprises between about 1.8E11 and about 6E11 vector genomes. In some embodiments, the second unit dose comprises about 1.8E11 vector genomes or about 6E11 vector genomes. In some embodiments, the first unit dose comprises about 2E11 vector genomes and the second unit dose comprises between about 6E11 and about 2E12 vector genomes. In some embodiments, the second unit dose comprises about 6E11 vector genomes or about 2E12 vector genomes. Additional details regarding unit doses are provided elsewhere herein. In some embodiments, the volume of the first unit dose is no more than about any one of 100, 95, 90, 85, 80, 75, 70, 65, 60, 55, 50, 45, 40, 35, 30, 25, 20, 15 or 10 μL. In some embodiments, the volume of the second unit dose is no more than about any one of 100, 95, 90, 85, 80, 75, 70, 65, 60, 55, 50, 45, 40, 35, 30, 25, 20, 15 or 10 μL.

Also provided is a method treating an ocular disease or disorder in a subject, comprising: administering a unit dose of a pharmaceutical composition to one eye of the subject via intravitreal (IVT) injection, wherein the pharmaceutical composition comprises: (a) a recombinant adeno-associated virus (rAAV) particle comprising a nucleic acid encoding a therapeutic protein, for example, an anti-vascular endothelial growth factor (VEGF) agent, wherein the rAAV particle is capable of infecting a retinal cell following IVT injection, and (b) a pharmaceutically acceptable excipient, and wherein the subject was administered with a prior unit dose of the pharmaceutical composition to a contralateral eye via IVT injection.

In some embodiments, the method further comprises a step of measuring a level of neutralizing antibodies against the rAAV in a sample from the subject following administration of the pharmaceutical composition to the contralateral eye and prior to the administration of pharmaceutical composition to the one eye. In some embodiments, the sample is a serum sample, a vitreous fluid sample, or an aqueous fluid sample. In some embodiments, measuring the level of nAbs comprises measuring the potency of neutralizing antibodies (nAbs) against the rAAV. In some embodiments, the neutralizing potency is quantified by the inhibitory concentration (IC), defined as the concentration of nAb (e.g., serum nAb) at which rAAV infectivity has been reduced by 50% relative to the absence of nAb (i.e., IC50). In some embodiments, rAAV particle infectivity is measured in an in vitro HEK293T cell-based assay. In some embodiments IC50 is expressed as the dilution factor necessary for a nAb-containing sample to cause a 50% reduction in rAAV particle infectivity. For example, where a nAb-containing sample required a 1:500 dilution to cause a 50% reduction in rAAV infectivity, the IC50 may be expressed as 500. In some embodiments, the IC50 is less than about any one of 600, 550, 500, 450, 400, 350, 300, 250, or 200. In some embodiments, the time interval between the step of administering the prior unit dose of the pharmaceutical composition to the contralateral eye and the step of administering the unit dose of the pharmaceutical composition to the one eye is at least about any one of 1, 2, 3, 4, 5, 6, 7, 8, 9, or 10 weeks, at least about any one of 1, 2, 3, 4, 5, 6, 7, 8, 9, 10, 11, or 12 months, or at least about any one of 0.5, 1, 1.5, 2, 2.5, 3, 3.5, 4, 4.5, 5, 5.5, 6, 6.5, 7, 7.5, 8, 8.5, 9, 10, 15, or 20 years, including any range between these values, and the step of measuring the level of neutralizing antibodies (nAbs) against the rAAV in the sample from the subject occurs within the time interval. In some embodiments, the level of neutralizing antibodies against the rAAV in a sample from the subject is measured via cut-point nAb assay, which is described elsewhere herein.

In some embodiments, the method further comprises a step of measuring expression level of the nucleic acid encoding the anti-VEGF agent in a sample from the subject following administration of the pharmaceutical composition to the contralateral eye and prior to the administration of pharmaceutical composition to the one eye. In some embodiments, the sample is a vitreous fluid sample or an aqueous fluid sample. In some embodiments, the expression level of the nucleic acid is measured by determining the abundance (e.g., relative abundance) of mRNA encoding the anti-VEGF agent. In some embodiments, the abundance (e.g., relative abundance) of mRNA is determined (e.g., quantified) via northern blot, RT-qPCR, RNA sequencing, RNA in situ hybridization, or other methods known in the art. In some embodiments, the expression level of the nucleic acid is measured by determining the abundance (e.g., relative abundance) of the anti-VEGF agent encoded by the nucleic acid. In some embodiments, the abundance (e.g., relative abundance) of anti-VEGF agent is determined (e.g., quantified) via western blot, Liquid chromatography-mass spectrometry (LC/MS), ELISA, immunohistochemistry, or other immunoassays known in the art. In some embodiments, the time interval between the step of administering the prior unit dose of the pharmaceutical composition to the contralateral eye and the step of administering the unit dose of the pharmaceutical composition to the one eye is at least about any one of 1, 2, 3, 4, 5, 6, 7, 8, 9, or 10 weeks; at least about any one of 1, 2, 3, 4, 5, 6, 7, 8, 9, 10, 11, or 12 months; or at least about any one of 0.5, 1, 1.5, 2, 2.5, 3, 3.5, 4, 4.5, 5, 5.5, 6, 6.5, 7, 7.5, 8, 8.5, 9, 10, 15, or 20 years, including any range between these values, and the step of measuring the expression level of the nucleic acid encoding the anti-VEGF agent in a sample from the subject occurs within the time interval.

In some embodiments the unit dose is a therapeutically effective dose, e.g., a dose sufficient to ensure efficient delivery of the nucleic acid (e.g., the nucleic acid encoding an anti-VEGF agent) into target cells (such as retinal) cells. In some embodiments, the prior unit dose was a therapeutically effective dose. In some embodiments, the unit dose and the prior unit dose are the same, e.g., between about 1E9 to about 3E13 vector genomes, between about 1E10 and about 1E13 vector genomes, between about 1E11 and 1E13 vector genomes, between about 1E10 to about 3E12 vector genomes, or between about 2E12 and about 6E12 vector genomes. In some embodiments, the unit dose is higher than the prior unit dose, e.g., at least about any one of 150%, 175%, 200%, 225%, 250%, 275%, or 300%, 350%, 400%, 450%, 500%, 550%, 600%, 650% 700%, 750%, 800%, 850%, 900%, 950%, or 1000% of the prior unit dose, including any range in between these values. For example, in some embodiments, the unit dose is at least about any one of 1.5-fold, 1.75-fold, 2-fold, 2.25-fold, 2.5-fold, 2.75-fold, 3-fold, 3.25-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, or 10-fold of the prior unit dose, including any range in between these values. In some embodiments, the prior unit dose comprised about 6E10 vector genomes and the unit dose comprises between about 1.8E11 and about 6E11 vector genomes. In some embodiments, the unit dose comprises about 1.8E11 vector genomes or about 6E11 vector genomes. In some embodiments, the prior unit dose comprised about 2E11 vector genomes and the unit dose comprises between about 6E11 and about 2E12 vector genomes. In some embodiments, the unit dose comprises about 6E11 vector genomes or about 2E12 vector genomes. Additional details regarding unit doses are provided elsewhere herein. In some embodiments, the volume of the unit dose is no more than about any one of 100, 95, 90, 85, 80, 75, 70, 65, 60, 55, 50, 45, 40, 35, 30, 25, 20, 15 or 10 μL. In some embodiments, the volume of the prior unit dose was no more than about any one of 100, 95, 90, 85, 80, 75, 70, 65, 60, 55, 50, 45, 40, 35, 30, 25, 20, 15 or 10 μL.

In some embodiments, the rAAV particle administered to the subject to treat and ocular disease or disorder comprises a variant capsid protein, wherein the variant capsid protein comprises an insertion of a peptide in the capsid protein GH loop relative to a corresponding parental capsid protein, wherein the insertion comprises an amino acid sequence selected from LALGETTRPA (SEQ ID NO: 1); LANETITRPA (SEQ ID NO: 2), LAKAGQANNA (SEQ ID NO: 3), LAKDPKTTNA (SEQ ID NO: 4), KDTDTTR (SEQ ID NO: 5), RAGGSVG (SEQ ID NO: 6), AVDTTKF (SEQ ID NO: 7), STGKVPN (SEQ ID NO: 8), LAKDTDTTRA (SEQ ID NO: 9), LARAGGSVGA (SEQ ID NO: 10), LAAVDTTKFA (SEQ ID NO: 11), and LASTGKVPNA (SEQ ID NO: 12). In some embodiments, the insertion site is within amino acids 570-611 of the AAV2 capsid protein set forth in SEQ ID NO: 13, or the corresponding position in the capsid protein of another AAV serotype. In some embodiments, the rAAV particle administered to the subject to treat and ocular disease or disorder is an rAAV2 particle comprising the amino acid sequence LALGETTRPA (SEQ ID NO: 1) inserted between positions 587 and 588 of SEQ ID NO: 13. The amino acid sequence of SEQ ID NO: 13 is provided below:

(SEQ ID NO: 13)
MAADGYLPDW LEDTLSEGIR QWWKLKPGPP PPKPAERHKD
DSRGLVLPGY KYLGPFNGLD KGEPVNEADA AALEHDKAYD
RQLDSGDNPY LKYNHADAEF QERLKEDTSF GGNLGRAVFQ
AKKRVLEPLG LVEEPVKTAP GKKRPVEHSP VEPDSSSGTG
KAGQQPARKR LNFGQTGDAD SVPDPQPLGQ PPAAPSGLGT
NTMATGSGAP MADNNEGADG VGNSSGNWHC DSTWMGDRVI
TTSTRTWALP TYNNHLYKQI SSQSGASNDN HYFGYSTPWG
YFDFNRFHCH FSPRDWQRLI NNNWGFRPKR LNFKLFNIQV
KEVTQNDGTT TIANNLTSTV QVFTDSEYQL PYVLGSAHQG
CLPPFPADVF MVPQYGYLTL NNGSQAVGRS SFYCLEYFPS
QMLRTGNNFT FSYTFEDVPF HSSYAHSQSL DRLMNPLIDQ
YLYYLSRTNT PSGTTTQSRL QFSQAGASDI RDQSRNWLPG
PCYRQQRVSK TSADNNNSEY SWTGATKYHL NGRDSLVNPG
PAMASHKDDE EKFFPQSGVL IFGKQGSEKT NVDIEKVMIT
DEEEIRTTNP VATEQYGSVS TNLQRGNRQA ATADVNTQGV
LPGMVWQDRD VYLQGPIWAK IPHTDGHFHP SPLMGGFGLK
HPPPQILIKN TPVPANPSTT FSAAKFASFI TQYSTGQVSV
EIEWELQKEN SKRWNPEIQY TSNYNKSVNV DFTVDTNGVY
SEPRPIGTRY LTRNL

In some embodiments, the rAAV particle administered to the subject to treat and ocular disease or disorder is an rAAV2 particle comprising the amino acid sequence of SEQ ID NO: 46, which is provided below:

(SEQ ID NO: 46)
MAADGYLPDW LEDTLSEGIR QWWKLKPGPP PPKPAERHKD
DSRGLVLPGY KYLGPFNGLD KGEPVNEADA AALEHDKAYD
RQLDSGDNPY LKYNHADAEF QERLKEDTSF GGNLGRAVFQ
AKKRVLEPLG LVEEPVKTAP GKKRPVEHSP VEPDSSSGTG
KAGQQPARKR LNFGQTGDAD SVPDPQPLGQ PPAAPSGLGT
NTMATGSGAP MADNNEGADG VGNSSGNWHC DSTWMGDRVI
TTSTRTWALP TYNNHLYKQI SSQSGASNDN HYFGYSTPWG
YFDFNRFHCH FSPRDWQRLI NNNWGFRPKR LNFKLFNIQV
KEVTQNDGTT TIANNLTSTV QVFTDSEYQL PYVLGSAHQG
CLPPFPADVF MVPQYGYLTL NNGSQAVGRS SFYCLEYFPS
QMLRTGNNFT FSYTFEDVPF HSSYAHSQSL DRLMNPLIDQ
YLYYLSRTNT PSGTTTQSRL QFSQAGASDI RDQSRNWLPG
PCYRQQRVSK TSADNNNSEY SWTGATKYHL NGRDSLVNPG
PAMASHKDDE EKFFPQSGVL IFGKQGSEKT NVDIEKVMIT
DEEEIRTTNP VATEQYGSVS TNLQRGNLAL GETTRPARQA
ATADVNTQGV LPGMVWQDRD VYLQGPIWAK IPHTDGHFHP
SPLMGGFGLK HPPPQILIKN TPVPANPSTT FSAAKFASFI
TQYSTGQVSV EIEWELQKEN SKRWNPEIQY TSNYNKSVNV
DFTVDTNGVY SEPRPIGTRY LTRNL

In some embodiments, the rAAV particle administered to the subject to treat and ocular disease or disorder comprises a variant capsid protein, wherein the variant capsid protein comprises a peptide insertion relative to a corresponding parental AAV capsid protein, wherein the peptide insertion has an amino acid sequence selected from LALGETTRPA (SEQ ID NO: 1); LANETITRPA (SEQ ID NO: 2), LAKAGQANNA (SEQ ID NO: 3), LAKDPKTTNA (SEQ ID NO: 4), KDTDTTR (SEQ ID NO: 5), RAGGSVG (SEQ ID NO: 6), AVDTTKF (SEQ ID NO: 7), STGKVPN (SEQ ID NO: 8), LAKDTDTTRA (SEQ ID NO: 9), LARAGGSVGA (SEQ ID NO: 10), LAAVDTTKFA (SEQ ID NO: 11), and LASTGKVPNA (SEQ ID NO: 12), wherein the insertion site is located between two adjacent amino acids at a position between amino acids corresponding to amino acids 570 and 611 of VP1 of AAV2 or the corresponding position in the capsid protein of another AAV serotype.

In some embodiments, rAAV particle administered to the subject to treat and ocular disease or disorder comprises a variant capsid protein, wherein the variant capsid protein comprises a modified sequence comprising one or more amino acid substitutions within amino acid residues 570-579 relative to a parental AAV capsid protein, wherein the modified sequence comprises HKFKSGD (SEQ ID NO: 1), and wherein the amino acid residue numbering corresponds to an AAV5 VP1 capsid protein. In some embodiments, the parental AAV capsid protein is an AAV5 capsid protein or an AAV5 and AAV2 hybrid capsid protein. In some embodiments, the parental AAV capsid protein is an AAV2.5T capsid protein. In some embodiments, “AAV2.5T capsid protein” or “AAV2.5T variant” refers to a hybrid capsid containing regions from AAV2 and AAV5, described in U.S. Pat. No. 9,441,244, the disclosure of which is incorporated in its entirety. AAV2.5T is capable of transducing the retina when delivered subretinally, but not when injected intravitreally. AAV2.5T transduction may be blocked by the inner limiting membrane (ILM), which is enriched with heparin sulfate proteoglycan (HSPG). The surface-exposed domains of AAV2.5T are identical to that of AAV5 except for a single substitution of A to T in aa582 of AAV2.5T (aa581 of AAV5), a mutation which appears to increase infectivity in mammalian cells without impacting AAV5's typical sialic acid receptor binding. AAV5 and AAV2.5T have negligible heparin sulfate binding, whereas AAV2 has high affinity for heparin sulfate. In some embodiments, the parental AAV capsid protein is an AAV2.5T VP1 capsid protein. In some embodiments, the modified sequence comprises LAHKFKSGDA (SEQ ID NO: 3). In some embodiments, the rAAV is AAV2.5T.LSV. In some embodiments, “AAV2.5T.LSV” or “AAV2.5T.LSV variant” refers to a rAAV variant that comprises a variant capsid protein, wherein the variant capsid protein comprises a loop substitution variant, wherein the loop substitution variant comprises the amino acid loop sequence LAHKFKSGDA (SEQ ID NO: 3) at amino acid residues 570-579 relative to AAV2.5T, the parental AAV capsid protein. Further details regarding recombinant adeno-associated viral vectors that can be used with the methods of the present application are provided elsewhere herein

In some embodiments, the anti-VEGF agent is a bevacizumab, brolucizumab, or ranibizumab. In some embodiments, the anti-VEGF agent is a polypeptide that comprises an amino acid sequence having at least 80% homology to aflibercept. In some embodiments, the anti-VEGF agent is aflibercept.

In some embodiments, the retinal cell is a photoreceptor, a retinal ganglion cell, a Müller cell, a bipolar cell, an amacrine cell, a horizontal cell, or a retinal pigmented epithelium cell.

In some embodiments, the ocular disease or disorder is characterized by abnormal (e.g., excessive) angiogenesis or neovascularization. In some embodiments, the ocular disease or disorder is, e.g., choroidal neovascularization, neovascular (wet) age-related macular degeneration (wAMD), macular edema following retinal vein occlusion, diabetic macular edema (DME), or diabetic retinopathy associated with DME, retinal vein occlusion, or any other related ocular disease or disorder characterized by abnormal (e.g., excessive) neovascularization in a subject. In some embodiments, ocular disease or disorder is a disease or disorder that is responsive to treatment with aflibercept (EYLEA®). In some embodiments, methods described herein are used to treat an ocular disease or disorder that is responsive to the current standard of care or is responsive to at least one of the approved therapies for AMD, RVO, DME, or DR in patients with DME, such as aflibercept injection, ranibizumab injection, brolucizumab injection, or bevacizumab injection.

In some embodiments, the pharmaceutical composition comprises a vector capable of delivering a nucleic acid encoding a polypeptide comprising an amino acid sequence that has at least 80% homology (such as at least any one of about 80%, 85%, 90%, 95%, 96%, 97%, 98%, or 99% homology) to aflibercept to retinal cells. In some embodiments, the pharmaceutical composition comprises a recombinant adeno-associated viral vector rAAV2 variant comprising a variant capsid protein that comprises amino acid sequence LALGETTRPA (SEQ ID NO: 1) inserted between positions 587 and 588 of capsid protein VP1 (e.g., as set forth in SEQ ID NO: 13), and a nucleic acid encoding a polypeptide comprising an amino acid sequence that has at least 80% homology (such as at least any one of about 80%, 85%, 90%, 95%, 96%, 97%, 98%, or 99% homology) to aflibercept and a pharmaceutically acceptable excipient. In some embodiments, the pharmaceutical composition comprises a recombinant adeno-associated viral vector rAAV2 variant comprising a variant capsid protein that comprises the amino acid sequence of SEQ ID NO: 46, and a nucleic acid encoding a polypeptide comprising an amino acid sequence that has at least 80% homology (such as at least any one of about 80%, 85%, 90%, 95%, 96%, 97%, 98%, or 99% homology) to aflibercept and a pharmaceutically acceptable excipient. In some embodiments, the nucleic acid encodes aflibercept.

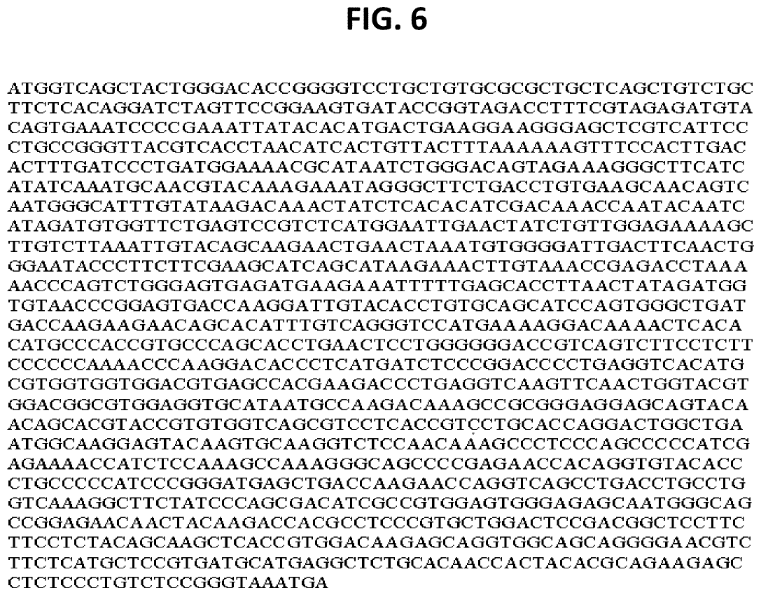

In some embodiments, the pharmaceutical composition comprises a recombinant adeno-associated viral vector rAAV2.5T variant comprising a variant capsid protein that comprises a loop substitution variant, wherein the loop substitution variant comprises the amino acid loop sequence LAHKFKSGDA (SEQ ID NO: 3) at amino acid residues 570-579 relative to AAV2.5T, the parental AAV capsid protein, and a nucleic acid encoding a polypeptide comprising an amino acid sequence that has at least 80% homology (such as at least any one of about 80%, 85%, 90%, 95%, 96%, 97%, 98%, or 99% homology) to aflibercept and a pharmaceutically acceptable excipient. In some embodiments, the nucleic acid encodes aflibercept. The protein sequence of aflibercept is publicly available at DrugBank database, accession number DB08885. In some embodiments, aflibercept refers to a nucleic acid sequence that encodes the protein, as disclosed in U.S. Patent Pub. 2014/0371438 (see, e.g., FIG. 6).

In some embodiments, the aflibercept (or functional fragment thereof or functional variant thereof) encoded by the nucleic acid delivered by the vector (e.g., recombinant adeno-associated viral vector) is expressed at a therapeutic dose in the target cells (e.g., retinal cells) of the first eye and the contralateral eye. In some embodiments, the aflibercept is expressed at a therapeutic dose in the first eye and the contralateral eye for at least about any one of 3, 4, 5, 6, 7, 8, 9, 10, 11, or 12 months, or for more than 12 months, e.g., at least about 1.5, 2, 2.5, 3, 3.5, 4, 4.5, 5, 5.5, 6, 6.5, 7, 7.5, 8, 8.5, 9, 9.5, 10, 15, or 20 years, including any range between any of these values, following administration via IVT injection to the first eye. In some embodiments, the aflibercept is expressed at a therapeutic dose in the first eye and the contralateral eye for at least about any one of 3, 4, 5, 6, 7, 8, 9, 10, 11, or 12 months, or for more than 12 months, e.g., at least about any one of 1.5, 2, 2.5, 3, 3.5, 4, 4.5, 5, 5.5, 6, 6.5, 7, 7.5, 8, 8.5, 9, 9.5, 10, 15, or 20 years, including any range between any of these values, following administration via IVT injection to the contralateral eye.

In some embodiments, the subject is a human or a non-human primate (e.g., an African green monkey or a rhesus macaque). In some embodiments, the subject is responsive to treatment with aflibercept. In some embodiments, the subject has been pre-treated (e.g., has received prior treatment) with aflibercept. The volume of a human eye is approximately two times that of the eye of a non-human primate (e.g., African green monkey), the dose (e.g., the number of vector genomes) administered to a first eye of a human is about 2× the dose (e.g., number of vector genomes) administered to a first eye of a non-human primate, and the dose (e.g., the number of vector genomes) administered to a second (e.g., contralateral) eye of a human is about 2× the dose (e.g., number of vector genomes) administered to a second (e.g., contralateral) eye of a non-human primate.

### Unit Doses/Therapeutically Effective Doses

In some embodiments, a unit dose comprises a therapeutically effective amount of vector (e.g., a viral vector). In some embodiments, a therapeutically effective amount of viral vector is an amount sufficient to ensure efficient delivery of the nucleic acid encoding, e.g., an anti-VEGF agent (such as aflibercept), into target cells (such as retinal cells). In some embodiments, a therapeutically effective amount of viral vector is an amount sufficient to ensure that the anti-VEGF agent (such as aflibercept) is delivered to and expressed in the target cells (e.g., retinal cells) at a level that produces a therapeutic effect. Exemplary therapeutic effects of treatment with the anti-VEGF agent (such as aflibercept) include, but are not limited to, the prevention of or delay in the development of one or more symptoms of the ocular disease, alter the course of a symptom disease, slowing the progression of one or more symptoms of the ocular disease, or reversing one or more symptoms of the ocular disease. Methods of monitoring the progression of ocular diseases described elsewhere herein are well known and widely used.

In some embodiments the unit dose (e.g., therapeutically effective amount) is expressed as the number of vector genomes administered to the subject. In some embodiments, a unit dose (e.g., therapeutically effective amount) of viral vector is between about 1×10¹⁰ to about 2×10¹⁰, between about 2×10¹⁰ to about 3×10¹⁰, between about 3×10¹⁰ to about 4×10¹⁰, between about 4×10¹⁰ to about 5×10¹⁰, between about 5×10¹⁰ to about 6×10¹⁰, between about 6×10¹⁰ to about 7×10¹⁰, between about 7×10¹⁰ to about 8×10¹⁰, between about 8×10¹⁰ to about 9×10¹⁰, between about 9×10¹⁰ to about 10×10¹⁰, between about 1×10¹¹ to about 2×10¹¹, between about 2×10¹¹ to about 3×10¹¹, between about 3×10¹¹ to about 4×10¹¹, between about 4×10¹¹ to about 5×10¹¹, between about 5×10¹¹ to about 6×10¹¹, between about 6×10¹¹ to about 7×10¹¹, between about 7×10¹¹ to about 8×10¹¹, between about 8×10¹¹ to about 9×10¹¹, between about 9×10¹¹ to about 10×10¹¹, between about 1×10¹² to about 2×10¹², between about 2×10¹² to about 3×10¹², between about 3×10¹² to about 4×10¹², between about 4×10¹² to about 5×10¹², between about 5×10¹² to about 6×10¹², between about 6×10¹² to about 7×10¹², between about 7×10¹² to about 8×10¹², between about 8×10¹² to about 9×10¹², between about 9×10¹² to about 10×10¹², between about 1×10¹³ to about 2×10¹³, between about 2×10¹³ to about 3×10¹³, between about 3×10¹³ to about 4×10¹³, between about 4×10¹³ to about 5×10¹³, between about 5×10¹³ to about 6×10¹³, between about 6×10¹³ to about 7×10¹³, between about 7×10¹³ to about 8×10¹³, between about 8×10¹³ to about 9×10¹³, or between about 9×10¹³ to about 10×10¹³ vector genomes, including any range in between these values.

In some embodiments, the unit dose (e.g., therapeutically effective amount) of viral vector (e.g., an rAAV vector disclosed herein) is about 2.1×10¹² vector genomes. In some embodiments, the unit dose (e.g., therapeutically effective amount) of viral vector (e.g., an rAAV vector disclosed herein) is between about 2×10¹² to about 6×10¹² vector genomes. In some embodiments, the unit dose (e.g., therapeutically effective amount) of viral vector (e.g., an rAAV vector disclosed herein) is between about 10¹⁰ to about 10¹³, between about 10¹⁰ to about 10¹¹, between about 10¹¹ to about 10¹², between about 10¹² to about 10¹³, between about 10¹³ to about 10¹⁴, between about 2×10¹¹ to about 4×10¹¹, between about 3×10¹¹ to about 5×10¹¹, between about 4×10¹¹ to about 6×10¹¹, between about 5×10¹¹ to about 7×10¹¹, between about 6×10¹¹ to about 8×10¹¹, between about 7×10¹¹ to about 9×10¹¹, between about 8×10¹¹ to about 10×10¹¹, between about 1×10¹² to about 3×10¹², between about 2×10¹² to about 4×10¹², between about 3×10¹² to about 5×10¹², between about 4×10¹² to about 6×10¹², between about 5×10¹² to about 7×10¹², between about 6×10¹² to about 8×10¹², between about 7×10¹² to about 9×10¹², between about 8×10¹² to about 10×10¹², between about 1×10¹³ to about 5×10¹³, between about 5×10¹³ to about 1×10¹⁴, between about 10¹² to about 5×10¹², or between about 5×10¹² to about 1×10¹³ vector genomes, including any range in between these values. In some embodiments, the unit dose (e.g., therapeutically effective amount) of viral vector (e.g., a rAAV vector disclosed herein such as rAAV2.7m8 or AAV2.5T.LSV) is between about 1×10¹⁰ to about 1×10¹³ vector genomes. In some embodiments, the unit dose (e.g., therapeutically effective amount) of viral vector (e.g., an rAAV vector disclosed herein) is about 1×10⁹ to about 1×10¹⁴ vector genomes.

In some embodiments, the unit dose (e.g., therapeutically effective amount) of viral vector (e.g., an rAAV vector disclosed herein such as rAAV2.7m8) is about 1×10¹⁰ to about 1×10¹¹ vector genomes. In some embodiments, the unit dose (e.g., therapeutically effective amount) of viral vector (e.g., a rAAV vector disclosed herein such as rAAV2.7m8) is be about 1×10⁸ to about 1×10¹⁵ vector genomes. In some embodiments, the unit dose (e.g., therapeutically effective amount) of viral vector (e.g., a rAAV vector disclosed herein such as rAAV2.7m8) is at least about any one of 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷ and 1×10¹⁸ vector genomes, including any range between these values. In some embodiments, the unit dose (e.g., therapeutically effective amount) of viral vector (e.g., an rAAV vector disclosed herein such as rAAV2.7m8) is 1×10⁸ to 1×10¹⁵ vector genomes. In some embodiments, the unit dose (e.g., therapeutically effective amount) of viral vector (e.g., a rAAV vector disclosed herein such as rAAV2.7m8) is at most about any one of 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷ and 1×10¹⁸ vector genomes. In some embodiments, the unit dose (e.g., therapeutically effective amount) of viral vector (e.g., a rAAV vector disclosed herein such as rAAV2.7m8) is between 10¹⁰ to 10¹¹, between 10¹¹ to 10¹², between 10¹⁰ to 10¹², between 10¹² to 10¹³, between 10¹¹ to 10¹³, between 10¹² to 10¹³, between 10¹² to 10¹⁴, between 10¹¹ to 10¹⁴, between 10¹¹ to 10¹⁵, between 10¹² to 10¹⁵, between 10¹³ to 10¹⁴, between 10¹⁴ to 10¹⁵, between 10¹⁵ to 10¹⁶, between 10¹⁶ to 10¹⁷, between 10¹⁷ to 10¹⁸, between 10¹⁸ to 10¹⁹, or between 10¹⁹ to 10²⁰ vector genomes, including any range in between these values.

In some embodiments, the unit dose (e.g., therapeutically effective amount) of viral vector (e.g., a rAAV vector disclosed herein such as rAAV2.7m8) is between about 1×10¹⁰ to 2×10¹⁰, between 2×10¹⁰ to 3×10¹⁰, between 3×10¹⁰ to 4×10¹⁰, between 4×10¹⁰ to 5×10¹⁰, between 5×10¹⁰ to 6×10¹⁰, between 6×10¹⁰ to 7×10¹⁰, between 7×10¹⁰ to 8×10¹⁰, between 8×10¹⁰ to 9×10¹⁰, between 9×10¹⁰ to 10×10¹⁰, between 1×10¹¹ to 2×10¹¹, between 2×10¹¹ to 3×10¹¹, between 2×10¹¹ to 2.5×10¹¹, between 2.5×10¹¹ to 3×10¹¹, between 3×10¹¹ to 4×10¹¹, between 4×10¹¹ to 5×10¹¹, between 5×10¹¹ to 6×10¹¹, between 6×10¹¹ to 7×10¹¹, between 7×10¹¹ to 8×10¹¹, between 8×10¹¹ to 9×10¹¹, between 9×10¹¹ to 10×10¹¹, between 1×10¹² to 2×10¹², between 2×10¹² to 3×10¹², between 2.5×10¹² to 3×10¹², between 3×10¹² to 4×10¹², between 4×10¹² to 5×10¹², between 5×10¹² to 6×10¹², between 6×10¹² to 7×10¹², between 7×10¹² to 8×10¹², between 8×10¹² to 9×10¹², between 9×10¹² to 10×10¹², between 1×10¹³ to 2×10¹³, between 2×10¹³ to 3×10¹³, between 3×10¹³ to 4×10¹³, between 4×10¹³ to 5×10¹³, between 5×10¹³ to 6×10¹³, between 6×10¹³ to 7×10¹³, between 7×10¹³ to 8×10¹³, between 8×10¹³ to 9×10¹³, or between 9×10¹³ to 10×10¹³ vector genomes.

In some embodiments, the unit dose (e.g., therapeutically effective amount) of viral vector (e.g., an rAAV vector disclosed herein) is between 2.1×10¹¹ or between 2.1×10¹² vector genomes. In some embodiments, the unit dose (e.g., therapeutically effective amount) of viral vector (e.g., an rAAV vector disclosed herein) is between 10¹⁰ to 10¹³, between 10¹⁰ to 10¹¹, between 10¹¹ to 10¹², between 10¹² to 10¹³, or between 10¹³ to 10¹⁴ vector genomes.

In some embodiments, the unit dose (e.g., therapeutically effective amount) of viral vector (e.g., an rAAV vector disclosed herein) is between 1×10¹⁰ to 2×10¹⁰, between 2×10¹⁰ to 4×10¹⁰, between 3×10¹⁰ to 5×10¹⁰, between 4×10¹⁰ to 6×10¹⁰, between 5×10¹⁰ to 7×10¹⁰, between 6×10¹⁰ to 8×10¹⁰, between 7×10¹⁰ to 9×10¹⁰, between 8×10¹⁰ to 10¹¹, between 1×10¹¹ to 2×10¹¹, between 2×10¹¹ to 4×10¹¹, between 3×10¹¹ to 5×10¹¹, between 4×10¹¹ to 6×10¹¹, between 5×10¹¹ to 7×10¹¹, between 6×10¹¹ to 8×10¹¹, between 7×10¹¹ to 9×10¹¹, between 8×10¹¹ to 10×10¹¹, between 1×10¹² to 3×10¹², between 2×10¹² to 4×10¹², between 3×10¹² to 5×10¹², between 4×10¹² to 6×10¹², between 5×10¹² to 7×10¹², between 6×10¹² to 8×10¹², between 7×10¹² to 9×10¹², between 8×10¹² to 10×10¹², between 1×10¹³ to 5×10¹³, between 5×10¹³ to 10×10¹³, between 10¹² to 5×10¹², between 5×10¹² to 1×10¹³, between 7×10¹² to 1×10¹³, between 8×10¹² to 2×10¹³, between 9×10¹² to 2×10¹³, between 9×10¹² to 2×10¹³, between 9×10¹² to 4×10¹³, between 1×10¹³ to 3×10¹³, between 1×10¹³ to 2×10¹³, between 2×10¹³ to 3×10¹³, between 3×10¹³ to 4×10¹³, between 4×10¹³ to 5×10¹³, between 5×10¹³ to 6×10¹³, between 6×10¹³ to 7×10¹³, between 7×10¹³ to 8×10¹³, between 8×10¹³ to 9×10¹³, or between 8×10¹³ to 1×10¹⁴ vector genomes.

In some embodiments, the unit dose (e.g., therapeutically effective amount) of vector genomes is selected from the lower range of values described herein in order to, e.g., avoid aggregation of the viral vector present in the pharmaceutical composition that is administered to the subject. In some embodiments, the unit dose (e.g., therapeutically effective amount) of vector genomes is selected from the higher range of values described herein in order to, e.g., ensure efficient delivery of the therapeutic transgene into target cells. In some embodiments, the unit dose (e.g., therapeutically effective dose) is selected from the higher range of values described herein in order to allow smaller volumes of injection, which can reduce adverse effects associated with intravitreal injection, e.g., elevated intraocular pressure, inflammation, irritation, or pain.

In some embodiments, the unit dose (e.g., therapeutically effective amount) of viral vector (e.g., a rAAV vector disclosed herein such as rAAV2.7m8) is about 1E10, about 1.5E10, about 2E10, about 2.5E10, about 3E10, about 3.5E10, about 4E10, about 4.5E10, about 5E10, about 5.5E10, about 6E10, about 6.5E10, about 7E10, about 7.5E10, about 8E10, about 8.5E10, about 9E10, about 9.5E10, about 1E11, about 1.5E11, about 2E11, about 2.5E11, about 3E11, about 3.5E11, about 4E11, about 4.5E11, about 5E11, about 5.5E11, about 6E11, about 6.5E11, about 7E11, about 7.5E11, about 8E11, about 8.5E11, about 9E11, about 9.5E11, about 1E12, about 1.3E12, about 1.5E12, about 2E12, about 2.1E12, about 2.3E12, about 2.5E12, about 2.7E12, about 2.9E12, about 3E12, about 3.1E12, about 3.3E12, about 3.5E12, about 3.7E12, about 3.9E12, about 4E12, about 4.1E12, about 4.3E12, about 4.5E12, about 4.7E12, about 4.9E12, about 5E12, about 5.1E12, about 5.3E12, about 5.5E12, about 5.7E12, about 5.9E12, about 6E12, about 6.1E12, about 6.3E12, about 6.5E12, about 6.7E12, about 6.9E12, about 7E12, about 7.1E12, about 7.3E12, about 7.5E12, about 7.7E12, about 7.9E12, about 8E12, about 8.1E12, about 8.3E12, about 8.5E12, about 8.7E12, about 8.9E12, about 9E12, about 9.1E12, about 9.3E12, about 9.5E12, about 9.7E12, about 9.9E12, about 1.01E13, about 1.03E13, about 1.05E13, about 1.07E13, about 1.09E13, about 1.1E13, about 1.15E13, about 1.2E13, about 1.25E13, about 1.3E13, about 1.35E13, about 1.4E13, about 1.45E13, about 1.5E13, about 1.55E13, about 1.6E13, about 1.65E13, about 1.7E13, about 1.75E13, about 1.8E13, about 1.85E13, about 1.9E13, about 1.95E13, about 2.0E13, about 2.5E13, about 3.0E13, about 3.5E13, about 4.0E13, about 4.5E13, about 5.0E13, about 5.5E13, about 6.0E13, about 6.5E13, about 7.0E13, about 7.5E13, about 8.0E13, about 8.5E13, about 9.0E13, about 9.5E13, or about 1E14 vector genomes, including any range in between these values, wherein E is a short-hand for base 10 for exponentiation, and xEy refers to x multiplied by base 10 to the y power/exponent. In some embodiments, the unit dose is (such as comprises) between about 1E9 to about 3E13 vector genomes, between about 1E10 and about 1E13 vector genomes, between about 1E11 and 1E13 vector genomes, between about 1E10 to about 3E12 vector genomes, or between about 2E12 and about 6E12 vector genomes.

In some embodiments, the unit dose (e.g., therapeutically effective amount) of viral vector is expressed as multiplicity of infection (MOI). In some cases, MOI refers to the ratio of vectors or viral genomes to the target cells to which the heterologous nucleic acid (e.g., the nucleic acid encoding the anti-VEGF agent (such as aflibercept)) is delivered. In some embodiments, the unit dose (e.g., therapeutically effective amount) of viral vector (e.g., an rAAV vector disclosed herein such as rAAV2.7m8) is an MOI of 1×10⁶. In some embodiments, the unit dose (e.g., therapeutically effective amount) of viral vector (e.g., a rAAV vector disclosed herein such as rAAV2.7m8) is an MOI of about any one of 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷ and 1×10¹⁸, including any range in between these values. In some embodiments, the unit dose (e.g., therapeutically effective amount) of viral vector (e.g., an rAAV vector disclosed herein such as rAAV2.7m8) is an MOI between about 1×10⁸ and about 1×10¹⁵. In some embodiments, a the unit dose (e.g., therapeutically effective amount) of viral vector (e.g., a rAAV vector disclosed herein such as rAAV2.7m8) is an MOI of no more than about any one of 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸, including any range in between these values. In some embodiments, a the unit dose (e.g., therapeutically effective amount) of viral vector (e.g., a rAAV vector disclosed herein such as rAAV2.7m8) is an MOI between 1×10¹⁰ to 2×10¹⁰, between 2×10¹⁰ to 4×10¹⁰, between 3×10¹⁰ to 5×10¹⁰, between 4×10¹⁰ to 6×10¹⁰, between 5×10¹⁰ to 7×10¹⁰, between 6×10¹⁰ to 8×10¹⁰, between 7×10¹⁰ to 9×10¹⁰, between 8×10¹⁰ to 10¹¹, between 1×10¹¹ to 2×10¹¹, between 2×10¹¹ to 4×10¹¹, between 3×10¹¹ to 5×10¹¹, between 4×10¹¹ to 6×10¹¹, between 5×10¹¹ to 7×10¹¹, between 6×10¹¹ to 8×10¹¹, between 7×10¹¹ to 9×10¹¹, between 8×10¹¹ to 10×10¹¹, between 1×10¹² to 3×10¹², between 2×10¹² to 4×10¹², between 3×10¹² to 5×10¹², between 4×10¹² to 6×10¹², between 5×10¹² to 7×10¹², between 6×10¹² to 8×10¹², between 7×10¹² to 9×10¹², between 8×10¹² to 10×10¹², between 1×10¹³ to 5×10¹³, between 5×10¹³ to 10×10¹³, between 10¹² to 5×10¹², between 5×10¹² to 1×10¹³, between 7×10¹² to 1×10¹³, between 8×10¹² to 2×10¹³, between 9×10¹² to 2×10¹³, between 9×10¹² to 2×10¹³, between 9×10¹² to 4×10¹³, between 1×10¹³ to 3×10¹³, between 1×10¹³ to 2×10¹³, between 2×10¹³ to 3×10¹³, between 3×10¹³ to 4×10¹³, between 4×10¹¹ to 5×10¹³, between 5×10¹³ to 6×10¹³, between 6×10¹³ to 7×10¹³, between 7×10¹³ to 8×10¹³, between 8×10¹³ to 9×10¹³, or between 8×10¹³ to 1×10¹⁴, including any range between these values.

In some embodiments, a unit dose (e.g., therapeutically effective amount) of viral vector is expressed as pfu (plaque forming units). In some embodiments, a unit dose (e.g., therapeutically effective amount) of viral vector (e.g., an rAAV vector disclosed herein such as rAAV2.7m8) is between about 1×10⁸ to about 1×10¹² pfu. In some embodiments, a unit dose (e.g., therapeutically effective amount) of viral vector (e.g., a rAAV vector disclosed herein such as rAAV2.7m8) is least about any one of 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹ or 1×10¹² pfu, including any range in between these values. In some embodiments, a unit dose (e.g., therapeutically effective amount) of viral vector (e.g., a rAAV vector disclosed herein such as rAAV2.7m8) is about any one of 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹ or 1×10¹² pfu, including any range in between these values.

In some embodiments, a therapeutically effective amount of viral vector is the amount sufficient to cause expression of the therapeutic protein (e.g., an anti-VEGF agent such as aflibercept) in the vitreous fluid to a achieve a concentration of at about any one of 0.3, 0.35, 0.4, 0.45, 0.5, 0.55, 0.6, 0.65, 0.7, 0.75, 0.8, 0.85, 0.5, 0.95, or 1.0 μg/ml, including any range in between these values. In some embodiments, a therapeutically effective amount of viral vector is the amount sufficient to cause expression of the therapeutic protein (e.g., an anti-VEGF agent such as aflibercept) in the retina to achieve a concentration of at least about 0.5, 0.55, 0.6, 0.65, 0.7, 0.75, 0.8, 0.85, 0.9, 0.95, or 1.0 μg/ml, including any range in between these values. In some embodiments, a therapeutically effective amount of viral vector is the amount sufficient to cause expression of the therapeutic protein (e.g., an anti-VEGF agent such as aflibercept) in the choroid to achieve a concentration of at about any one of 0.3, 0.35, 0.4, 0.45, 0.5, 0.55, 0.6, 0.65, 0.7, 0.75, 0.8, 0.85, 0.5, 0.95, or 1.0 μg/ml, including any range in between these values.

In some embodiments, the dose (e.g., unit dose) of viral vector administered to the subject is therapeutically effective if administration of the dose to the subject reduces, stops, or prevents at least one symptom of the ocular disease or disorder. In the cases of ocular diseases or disorders characterized by abnormal (e.g., excessive) angiogenesis, such symptoms include, but are not limited to, e.g., visual distortions (such as impaired color vision, blurred vision, deterioration of central vision) and vision loss. In some embodiments, the dose (e.g., unit dose) of viral vector administered to the subject is therapeutically effective if administration of the dose to the subject results in the maintenance, partial resolution, or complete resolution of one or more clinical features of the disease. For example, the dose (e.g., unit dose) of viral vector administered to the subject is therapeutically effective if administration of the dose to the subject results in (a) complete resolution, partial resolution or maintenance of the ocular disease as measured by optical coherence tomography (OCT); (b) an increase and/or maintenance in best corrected visual acuity (such as assessed by an EDTRS eye chart, Amsler grid, etc.); (c) maintenance or reduction of hyperfluorescence as measured via fluorescein angiography (FA).

### Exemplary Vectors Delivering Transgenes to Target Cells

In some embodiments, delivery of a heterologous nucleic acid encoding an anti-VEGF agent (e.g., aflibercept) to a target cell (e.g., a retinal cell) is performed using any suitable vector (also referred to as “gene delivery” or “gene transfer vehicle”). In some embodiments, the vector, delivery vehicle, gene delivery vehicle, or gene transfer vehicle is a macromolecule or complex of molecules that comprises a heterologous nucleic acid and is capable of delivering the heterologous nucleic acid to a target cell. In some embodiments, the target cell is a retinal cell, e.g., any of the cell types that comprise the retina, such as a photoreceptor, a retinal ganglion cell, a Müller cell, a bipolar cell, an amacrine cell, a horizontal cell, or a retinal pigmented epithelium cell). In some embodiments, the target cell is any cell to which the nucleic acid molecule or gene is delivered. In some embodiments, the heterologous nucleic acid encodes an anti-VEGF agent (e.g., aflibercept). In some embodiments, the vector, delivery vehicle, gene delivery vehicle, or gene transfer vehicle is present in a pharmaceutical composition that is formulated or adapted for administration to the eye of a subject (e.g., a human or non-human primate) via intravitreal injection (IVT).

In some embodiments, the vector is a viral vector, e.g., an adenovirus, an adeno-associated virus (AAV), a retrovirus, or a lentivirus, or a hybrid viral vector. In some embodiments the viral vector is a recombinant viral vector. In some embodiments, the viral vector (e.g., recombinant viral vector) comprises a heterologous nucleic acid (e.g., a heterologous nucleic acid encoding an anti-VEGF agent such as aflibercept) that is operably linked to a strong eukaryotic promoter (e.g., a cytomegalovirus (CMV) promoter or a constitutive promoter). In some embodiments, the viral vector (such as a recombinant viral vector) comprises at least one (such as more than one) nucleic acid molecule. In some embodiments, the at least one (such as more than one) nucleic acid is a DNA (e.g., cDNA) or an RNA. In some embodiments, viral vector (such as a recombinant viral vector) comprises both DNA and RNA. In some embodiments, the RNA is a transcript (e.g., a transcript of an anti-VEGF agent such as aflibercept) that comprises, e.g., introns, untranslated regions (UTRs), termination sequences and the like. In some embodiments, the DNA encodes an anti-VEGF agent (e.g., aflibercept) and optionally further comprises promoter sequences, UTRs, termination sequences, and the like. In some embodiments, the vector is a recombinant adeno-associated virus (rAAV) capable of delivering a heterologous nucleic acid (e.g., a nucleic acid encoding an anti-VEGF agent such as aflibercept) to a target cell (e.g., a retinal cell) in which the heterologous nucleic acid is expressed. In some embodiments, expression of the heterologous nucleic acid exerts therapeutic effect in the target tissue.

In some embodiments, the vector is a recombinant viral vector derived from adenovirus (Ad) or adeno-associated virus (AAV) that has been altered so that it is replication-defective in the subject (e.g., a human or a non-human primate). In some embodiments the adeno-associated virus (AAV) is a recombinant AAV (rAAV). In some embodiments, the heterologous nucleic acid (e.g., a nucleic acid that encodes an anti-VEGF agent such as aflibercept) integrates into the target cell genome (e.g., retinal cell genome), resulting in long-term expression of, e.g., the anti-VEGF agent (such as aflibercept), in the target cell. In some embodiments, the viral vector delivers a plasmid or other extrachromosomal genetic element that comprises the heterologous nucleic acid (e.g., a nucleic acid that encodes an anti-VEGF agent such as aflibercept) to the target cell (e.g., retinal cell).

AAV or rAAV are small non-enveloped single-stranded DNA viruses. rAAVs are non-pathogenic human parvoviruses and can be made to be dependent on helper viruses, including adenovirus, herpes simplex virus, vaccinia virus and CMV, for replication. Exposure to wild-type (wt) AAV is not associated or known to cause any human pathologies and is common in the general population, making AAV or rAAV a suitable delivery system for gene therapy. AAV and rAAV used for gene therapy for delivery of an anti-VEGF agent, e.g., aflibercept, can be of any serotype. In some embodiments, pharmaceutical compositions and methods of the disclosure provide for use of any suitable AAV serotype, including AAV1, AAV2, AAV2.5, AAV3, AAV4, AAV5, AAV6, AAV7, AAV8, AAV9, AAV10, AAV11, AAV12, rh10, AAV-DJ, and any hybrid or chimeric AAV thereof. In some embodiments, the serotype used is based on tropism of the virus, or infectivity of a target cell of interest. In some embodiments, AAV2 or rAAV2 is used to deliver a nucleic acid sequence encoding an anti-VEGF agent (e.g., aflibercept) into an eye or retinal cells of a subject via intravitreal or subretinal injection. In some embodiments, rAAV2.7m8 is used to deliver the nucleic acid sequence of the anti-VEGF agent (e.g., aflibercept) into the retinal cells of a subject.

In some embodiments, AAV or rAAV viruses, particles, or virions comprising a variant capsid protein having increased infectivity of target cells, e.g. retinal cells, are used to increase transduction of retinal cells or to increase targeting of gene delivery to retinal cells in a subject. In some embodiments, the rAAV particle comprises an amino acid modification in a capsid protein GH loop/loop IV of the AAV capsid protein. In some embodiments, the site of modification is a solvent-accessible portion of the GH loop/loop IV of the AAV capsid protein. For a description of the GH loop/loop IV of AAV capsid, see, e.g., van Vliet et al. (2006) Mol. Ther. 14:809; Padron et al. (2005) J. Virol. 79:5047; and Shen et al. (2007) Mol. Ther. 15:1955. Several AAV capsid variants are known, including the 7m8 variant. In some embodiments, a rAAV particle comprises a variant AAV capsid protein that comprises an insertion of from 5 amino acids to 11 amino acids, e.g., 7 amino acid sequence, in the GH loop of a capsid protein relative to a corresponding parental AAV capsid protein, and wherein the variant capsid protein confers increased infectivity of a retinal cell compared to the infectivity of the retinal cell by an AAV particle comprising the corresponding parental or unmodified AAV capsid protein. In some embodiments, any one of the following amino acid sequences can be inserted in the GH loop of a capsid protein: LALGETTRPA (SEQ ID NO: 1); LANETITRPA (SEQ ID NO: 2), LAKAGQANNA (SEQ ID NO: 3), LAKDPKTTNA (SEQ ID NO: 4), KDTDTTR (SEQ ID NO: 5), RAGGSVG (SEQ ID NO: 6), AVDTTKF (SEQ ID NO: 7), STGKVPN (SEQ ID NO: 8), LAKDTDTTRA (SEQ ID NO: 9), LARAGGSVGA (SEQ ID NO: 10), LAAVDTTKFA (SEQ ID NO: 11), and LASTGKVPNA (SEQ ID NO: 12), LGETTRP (SEQ ID NO: 14), NETITRP (SEQ ID NO: 15), KAGQANN (SEQ ID NO: 16), KDPKTTN (SEQ ID NO: 17), KDTDTTR (SEQ ID NO: 18), RAGGSVG (SEQ ID NO: 19), AVDTTKF (SEQ ID NO: 20), and STGKVPN (SEQ ID NO: 21). In some embodiments, any one of the amino acid sequences set forth in SEQ ID NOs: 1-12 and 14-21 is inserted in the solvent-exposed GH loop of VP1 capsid protein in a rAAV. Additional details regarding amino acid sequences that can be inserted into the GH loop of a capsid protein, e.g., to facilitate transduction of a nucleic acid of interest to a retinal cell following IVT injection, are provided in WO2012145601, the contents of which are incorporated herein by reference in their entirety. In some embodiments, rAAV.7m8 comprising aflibercept is used for gene therapy.

In some embodiments, any one of the following amino acid sequences: LALGETTRPA (SEQ ID NO: 1); LANETITRPA (SEQ ID NO: 2), LAKAGQANNA (SEQ ID NO: 3), LAKDPKTTNA (SEQ ID NO: 4), KDTDTTR (SEQ ID NO: 5), RAGGSVG (SEQ ID NO: 6), AVDTTKF (SEQ ID NO: 7), STGKVPN (SEQ ID NO: 8), LAKDTDTTRA (SEQ ID NO: 9), LARAGGSVGA (SEQ ID NO: 10), LAAVDTTKFA (SEQ ID NO: 11), and LASTGKVPNA (SEQ ID NO: 12), LGETTRP (SEQ ID NO: 14), NETITRP (SEQ ID NO: 15), KAGQANN (SEQ ID NO: 16), KDPKTTN (SEQ ID NO: 17), KDTDTTR (SEQ ID NO: 18), RAGGSVG (SEQ ID NO: 19), AVDTTKF (SEQ ID NO: 20), and STGKVPN (SEQ ID NO: 21) can be inserted at the following positions to generate a rAAV variant for use in gene therapy: between positions 587 and 588 of the AAV2 capsid protein; between amino acids 590 and 591 of the AAV1 capsid protein; between amino acids 575 and 576 of the AAV5 capsid protein; between amino acids 590 and 591 of the AAV6 capsid protein; between amino acids 589 and 590 of the AAV7 capsid protein; between amino acids 590 and 591 of the AAV8 capsid protein; between amino acids 588 and 589 of the AAV9 capsid protein; or between amino acids 589 and 590 of the AAV10 capsid protein.

In some embodiments, AAV or rAAV viruses, particles, or virions comprising a variant capsid protein that are used in gene therapy exhibit one or more of the following characteristics: 1) increased infectivity of a retinal cell; 2) altered tropism; 3) increased binding to heparin and/or heparin sulfate proteogly cans and/or the inner limiting membrane (ILM); and 4) an increased ability to infect and/or deliver a therapeutic gene product across the ILM when administered intravitreally, as compared to a corresponding viral vector comprising its native, wild-type, and/or parental capsid protein. In some embodiments, the variant AAV capsid protein used in gene therapy comprises a modified sequence comprising one or more amino acids substitutions within amino acid residues 570-579 relative to a parental AAV capsid protein, wherein the modified sequence comprises HKFKSGD (SEQ ID NO: 37), and wherein the amino acid residue numbering corresponds to an AAV5 VP1 capsid protein. In some embodiments, the variant AAV capsid protein comprises a modified sequence comprising one or more amino acids substitutions within amino acid residues 570-579 relative to a parental AAV capsid protein, wherein the modified sequence comprises X₁X₂HKFKSGDX₃ (SEQ ID NO:38), and wherein the amino acid residue numbering corresponds to an AAV5 VP1 capsid protein, wherein X₁₋₃ can independently be any amino acid. In some embodiments, each of X₁₋₃ is independently selected from A, L, G, S, and T. In some embodiments, each of X₁₋₃ is independently selected from A, L, G, S, and T. In some embodiments, X₁ is L. In some embodiments, X₂ is A. In some embodiments, X₃ is A. In some embodiments, the variant AAV capsid protein comprises a modified sequence comprising one or more amino acids substitutions within amino acid residues 570-579 relative to a parental AAV capsid protein, wherein the modified sequence comprises LAHKFKSGDA (SEQ ID NO: 39), a sequence having at least 80% or at least 90% homology with SEQ ID NO: 39; having at least 80% or at least 90% sequence identity with SEQ ID NO: 39; or having four or more, five or more, six or more, seven or more, eight or more, or nine or more consecutive amino acids within SEQ ID NO: 39, and wherein the amino acid residue numbering corresponds to an AAV5 VP1 capsid protein. In some embodiments, the modified sequence comprises LAHKFKSGDA (SEQ ID NO: 39).

In some embodiments, the parental AAV capsid protein is a wild-type AAV capsid protein, for example an AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), avian AAV, bovine AAV, canine AAV, equine AAV, primate AAV, non-primate AAV, or ovine AAV capsid protein. In some embodiments, the parental AAV capsid protein is an AAV5 capsid protein. In some embodiments, the parental AAV capsid protein is a VP1, VP2, or VP3 capsid protein. In some embodiments, the parental AAV capsid protein is an AAV5 VP1 capsid protein.

In some embodiments, the parental AAV capsid protein is a variant AAV capsid protein. In some embodiments, the parental AAV capsid protein is a variant of an AAV1, an AAV2, an AAV3, an AAV4, an AAV5, an AAV6, an AAV7, an AAV8, an AAV9, an AAV10, an avian AAV, a bovine AAV, a canine AAV, an equine AAV, a primate AAV, a non-primate AAV, or an ovine AAV capsid protein. In some embodiments, the parental AAV capsid protein is a variant of an AAV5 VP1 capsid protein (SEQ ID NO: 40). In some embodiments, the parental AAV capsid protein is a variant of an AAV5 VP1 capsid protein having at least 90%, at least 95%, at least 98%, at least 99% homology to SEQ ID NO: 40. In some embodiments, the parental AAV capsid protein is a variant of an AAV5 VP1 capsid protein having at least 90%, at least 95%, at least 98%, at least 99% sequence identity to SEQ ID NO: 40. In some embodiments, the parental AAV capsid protein is a hybrid capsid protein. In some embodiments, the parental AAV capsid protein is a hybrid of AAV2 and AAV5. In some embodiments, the parental AAV capsid protein is AAV 2.5T, or a variant thereof having at least 90%, at least 95%, at least 98%, at least 99% homology to an AAV2.5T capsid protein. In some embodiments, the parental AAV capsid protein is AAV 2.5T, or a variant thereof having at least 90%, at least 95%, at least 98%, at least 99% sequence identity to an AAV2.5T capsid protein. In some embodiments, the parental AAV capsid protein is AAV 2.5T. In some embodiments, the parental AAV capsid protein is a VP1, VP2, or VP3 capsid protein. In some embodiments, the parental AAV capsid protein is an AAV2.5T VP1 capsid protein, or a variant thereof having at least 90%, at least 95%, at least 98%, at least 99% homology to an AAV2.5T VP1 capsid protein (SEQ ID NO: 41). In some embodiments, the parental AAV capsid protein is an AAV2.5T VP1 capsid protein, or a variant thereof having at least 90%, at least 95%, at least 98%, at least 99% sequence identity to an AAV2.5T VP1 capsid protein (SEQ ID NO: 41).

In some embodiments, the variant AAV capsid protein comprises a capsid sequence having at least 85%, homology to the amino acid sequence set forth in SEQ ID NO: 40 or SEQ ID NO: 41. In some embodiments, the variant AAV capsid protein comprises a capsid sequence having at least 85%, sequence identity to the amino acid sequence set forth in SEQ ID NO: 40 or SEQ ID NO: 41. In some embodiments, the variant AAV capsid protein comprises a capsid sequence having at least 85%, at least 90%, at least 95%, at least 98%, at least 99%, or 100% homology to the amino acid sequence set forth in SEQ ID NO: 42 (AAV5.LSV1 VP1) or SEQ ID NO: 43 (AAV2.5T.LSV1 VP1). In some embodiments, the variant AAV capsid protein comprises a capsid sequence having at least 85%, at least 90%, at least 95%, at least 98%, at least 99%, or 100% sequence identity to the amino acid sequence set forth in SEQ ID NO: 42 or SEQ ID NO: 43. In some embodiments, the variant AAV capsid protein comprises SEQ ID NO: 42 or SEQ ID NO: 43. In some embodiments, the variant AAV capsid protein comprises SEQ ID NO: 42. In some embodiments, the variant AAV capsid protein comprises SEQ ID NO: 43. The amino acid sequences of SEQ ID NOs: 40, 41, 42, and 43 are provided below:

(SEQ ID NO: 40)
MSFVDHPPDW LEEVGEGLRE FLGLEAGPPK PKPNQQHQDQ
ARGLVLPGYN YLGPGNGLDR GEPVNRADEV AREHDISYNE
QLEAGDNPYL KYNHADAEFQ EKLADDTSFG GNLGKAVFQA
KKRVLEPFGL VEEGAKTAPT GKRIDDHFPK RKKARTEEDS
KPSTSSDAEA GPSGSQQLQI PAQPASSLGA DTMSAGGGGP
LGDNNQGADG VGNASGDWHC DSTWMGDRVV TKSTRTWVLP
SYNNHQYREI KSGSVDGSNA NAYFGYSTPW GYFDFNRFHS
HWSPRDWQRL INNYWGFRPR SLRVKIFNIQ VKEVTVQDST
TTIANNLTST VQVFTDDDYQ LPYVVGNGTE GCLPAFPPQV
FTLPQYGYAT LNRDNTENPT ERSSFFCLEY FPSKMLRTGN
NFEFTYNFEE VPFHSSFAPS QNLFKLANPL VDQYLYRFVS
TNNTGGVQFN KNLAGRYANT YKNWFPGPMG RTQGWNLGSG
VNRASVSAFA TTNRMELEGA SYQVPPQPNG MTNNLQGSNT
YALENTMIFN SQPANPGTTA TYLEGNMLIT SESETQPVNR
VAYNVGGQMA TNNQSSTTAP ATGTYNLQEI VPGSVWMERD
VYLQGPIWAK IPETGAHFHP SPAMGGFGLK HPPPMMLIKN
TPVPGNITSF SDVPVSSFIT QYSTGQVTVE MEWELKKENS
KRWNPEIQYT NNYNDPQFVD FAPDSTGEYR TTRPIGTRYL
TRPL
(SEQ ID NO: 41)
MAADGYLPDW LEDTLSEGIR QWWKLKPGPP PPKPAERHKD
DSRGLVLPGY KYLGPFNGLD KGEPVNEADA AALEHDKAYD
RQLDSGDNPY LKYNHADAEF QERLKEDTSF GGNLGRAVFQ
AKKRVLEPFG LVEEGAKTAP TGKRIDDHFP KRKKARTEED
SKPSTSSDAE AGPSGSQQLQ IPAQPASSLG ADTMSAGGGG
PLGDNNQGAD GVGNASGDWH CDSTWMGDRV VTKSTRTWVL
PSYNNHQYRE IKSGSVDGSN ANAYFGYSTP WGYFDFNRFH
SHWSPRDWQR LINNYWGFRP RSLRVKIFNI QVKEVTVQDS
TTTIANNLTS TVQVFTDDDY QLPYVVGNGT EGCLPAFPPQ
VFTLPQYGYA TLNRDNTENP TERSSFFCLE YFPSKMLRTG
NNFEFTYNFE EVPFHSSFAP SQNLFKLANP LVDQYLYRFV
STNNTGGVQF NKNLAGRYAN TYKNWFPGPM GRTQGWNLGS
GVNRASVSAF ATTNRMELEG ASYQVPPQPN GMTNNLQGSN
TYALENTMIF NSQPANPGTT ATYLEGNMLI TSESETQPVN
RVAYNVGGQM ATNNQSSTTA PTTGTYNLQE IVPGSVWMER
DVYLQGPIWA KIPETGAHFH PSPAMGGFGL KHPPPMMLIK
NTPVPGNITS FSDVPVSSFI TQYSTGQVTV EMEWELKKEN
SKRWNPEIQY TNNYNDPQFV DFAPDSTGEY RTTRPIGTRY
LTRPL
(SEQ ID NO: 42)
MSFVDHPPDW LEEVGEGLRE FLGLEAGPPK PKPNQQHQDQ
ARGLVLPGYN YLGPGNGLDR GEPVNRADEV AREHDISYNE
QLEAGDNPYL KYNHADAEFQ EKLADDTSFG GNLGKAVFQA
KKRVLEPFGL VEEGAKTAPT GKRIDDHFPK RKKARTEEDS
KPSTSSDAEA GPSGSQQLQI PAQPASSLGA DTMSAGGGGP
LGDNNQGADG VGNASGDWHC DSTWMGDRVV TKSTRTWVLP
SYNNHQYREI KSGSVDGSNA NAYFGYSTPW GYFDFNRFHS
HWSPRDWQRL INNYWGFRPR SLRVKIFNIQ VKEVTVQDST
TTIANNLTST VQVFTDDDYQ LPYVVGNGTE GCLPAFPPQV
FTLPQYGYAT LNRDNTENPT ERSSFFCLEY FPSKMLRTGN
NFEFTYNFEE VPFHSSFAPS QNLFKLANPL VDQYLYRFVS
TNNTGGVQFN KNLAGRYANT YKNWFPGPMG RTQGWNLGSG
VNRASVSAFA TTNRMELEGA SYQVPPQPNG MTNNLQGSNT
YALENTMIFN SQPANPGTTA TYLEGNMLIT SESETQPVNR
VAYNVGGQML AHKFKSGDAP ATGTYNLQEI VPGSVWMERD
VYLQGPIWAK IPETGAHFHP SPAMGGFGLK HPPPMMLIKN
TPVPGNITSF SDVPVSSFIT QYSTGQVTVE MEWELKKENS
KRWNPEIQYT NNYNDPQFVD FAPDSTGEYR TTRPIGTRYL
TRPL
(SEQ ID NO: 43)
MAADGYLPDW LEDTLSEGIR QWWKLKPGPP PPKPAERHKD
DSRGLVLPGY KYLGPFNGLD KGEPVNEADA AALEHDKAYD
RQLDSGDNPY LKYNHADAEF QERLKEDTSF GGNLGRAVFQ
AKKRVLEPFG LVEEGAKTAP TGKRIDDHFP KRKKARTEED
SKPSTSSDAE AGPSGSQQLQ IPAQPASSLG ADTMSAGGGG
PLGDNNQGAD GVGNASGDWH CDSTWMGDRV VTKSTRTWVL
PSYNNHQYRE IKSGSVDGSN ANAYFGYSTP WGYFDFNRFH
SHWSPRDWQR LINNYWGFRP RSLRVKIFNI QVKEVTVQDS
TTTIANNLTS TVQVFTDDDY QLPYVVGNGT EGCLPAFPPQ
VFTLPQYGYA TLNRDNTENP TERSSFFCLE YFPSKMLRTG
NNFEFTYNFE EVPFHSSFAP SQNLFKLANP LVDQYLYRFV
STNNTGGVQF NKNLAGRYAN TYKNWFPGPM GRTQGWNLGS
GVNRASVSAF ATTNRMELEG ASYQVPPQPN GMTNNLQGSN
TYALENTMIF NSQPANPGTT ATYLEGNMLI TSESETQPVN
RVAYNVGGQM LAHKFKSGDA PTTGTYNLQE IVPGSVWMER
DVYLQGPIWA KIPETGAHFH PSPAMGGFGL KHPPPMMLIK
NTPVPGNITS FSDVPVSSFI TQYSTGQVTV EMEWELKKEN
SKRWNPEIQY TNNYNDPQFV DFAPDSTGEY RTTRPIGTRY
LTRPL

In some embodiments, the variant AAV capsid protein comprises a capsid sequence having at least 85%, at least 90%, at least 95%, at least 98%, at least 99%, or 100% homology to the amino acid sequence set forth in SEQ ID NO: 44 (AAV5.LSV1 VP2) or SEQ ID NO: 45 (AAV5.LSV1 VP3). In some embodiments, the variant AAV capsid protein comprises a capsid sequence having at least 85%, at least 90%, at least 95%, at least 98%, at least 99%, or 100% sequence identity to the amino acid sequence set forth in SEQ ID NO: 44 or SEQ ID NO: 45. In some embodiments, the variant AAV capsid protein comprises SEQ ID NO: 44 or SEQ ID NO: 45. In some embodiments, the variant AAV capsid protein comprises SEQ ID NO: 44. In some embodiments, the variant AAV capsid protein comprises SEQ ID NO: 45. The amino acid sequences of SEQ ID NOs: 44 and 45 are provided below:

(SEQ ID NO: 44)
APTGKRIDDH FPKRKKARTE EDSKPSTSSD AEAGPSGSQQ
LQIPAQPASS LGADTMSAGG GGPLGDNNQG ADGVGNASGD
WHCDSTWMGD RVVTKSTRTW VLPSYNNHQY REIKSGSVDG
SNANAYFGYS TPWGYFDFNR FHSHWSPRDW QRLINNYWGF
RPRSLRVKIF NIQVKEVTVQ DSTTTIANNL TSTVQVFTDD
DYQLPYVVGN GTEGCLPAFP PQVFTLPQYG YATLNRDNTE
NPTERSSFFC LEYFPSKMLR TGNNFEFTYN FEEVPFHSSF
APSQNLFKLA NPLVDQYLYR FVSTNNTGGV QFNKNLAGRY
ANTYKNWFPG PMGRTQGWNL GSGVNRASVS AFATTNRMEL
EGASYQVPPQ PNGMTNNLQG SNTYALENTM IFNSQPANPG
TTATYLEGNM LITSESETQP VNRVAYNVGG QMLAHKFKSG
DAPTTGTYNL QEIVPGSVWM ERDVYLQGPI WAKIPETGAH
FHPSPAMGGF GLKHPPPMML IKNTPVPGNI TSFSDVPVSS
FITQYSTGQV TVEMEWELKK ENSKRWNPEI QYTNNYNDPQ
FVDFAPDSTG EYRTTRPIGT RYLTRPL
(SEQ ID NO: 45)
MSAGGGGPLG DNNQGADGVG NASGDWHCDS TWMGDRVVTK
STRTWVLPSY NNHQYREIKS GSVDGSNANA YFGYSTPWGY
FDFNRFHSHW SPRDWQRLIN NYWGFRPRSL RVKIFNIQVK
EVTVQDSTTT IANNLTSTVQ VFTDDDYQLP YVVGNGTEGC
LPAFPPQVFT LPQYGYATLN RDNTENPTER SSFFCLEYFP
SKMLRTGNNF EFTYNFEEVP FHSSFAPSQN LFKLANPLVD
QYLYRFVSTN NTGGVQFNKN LAGRYANTYK NWFPGPMGRT
QGWNLGSGVN RASVSAFATT NRMELEGASY QVPPQPNGMT
NNLQGSNTYA LENTMIFNSQ PANPGTTATY LEGNMLITSE
SETQPVNRVA YNVGGQMLAH KFKSGDAPTT GTYNLQEIVP
GSVWMERDVY LQGPIWAKIP ETGAHFHPSP AMGGFGLKHP
PPMMLIKNTP VPGNITSFSD VPVSSFITQY STGQVTVEME
WELKKENSKR WNPEIQYTNN YNDPQFVDFA PDSTGEYRTT
RPIGTRYLTR PL

While reference above is made to amino acid modifications of capsid proteins (including specific amino acid substitutions and insertions) using the amino acid numbering corresponding to AAV5 VP1, VP2, or VP3 capsid protein, it is understood that any of these amino acid modifications may also be introduced in the capsid protein of AAVs of other serotypes, e.g., at positions corresponding to those of AAV5 VP1, VP2, or VP3. AAV protein sequences share significant homology and similar amino acid numbering, and the skilled artisan can readily determine amino acid residues in other AAV serotypes that correspond to those specifically described above for AAV5 VP1, VP2, and VP3. Additional details regarding variant AAV capsid proteins that comprise a modified sequence comprising one or more amino acids substitutions within amino acid residues 570-579 (e.g., HKFKSGD (SEQ ID NO: 37)) relative to a parental AAV capsid protein and their uses in gene therapy are detailed in U.S. Ser. No. 62/839,548 and U.S. Ser. No. 62/923,924, the contents of which are incorporated by reference herein in their entirety.

In some embodiments, the heterologous nucleic acid that encodes an anti-VEGF agent (e.g., aflibercept) is under the transcriptional control of a promoter that initiates transcription of the heterologous nucleic acid. In some embodiments, the promoter is a “strong” or constitutively active promoter, e.g., a cytomegalovirus (CMV) promoter, an elongation factor 1 alpha (EF1a) promoter, a glyceraldehyde 3-phosphate dehydrogenase (GAPDH) promoter, or a connexin36 (or “Cx36”) promoter. In some embodiments, the promoter is a tissue-specific promoter that is activated in specific tissues or cells, such as retinal cells, to reduce potential toxicity or undesirable effects to non-targeted cells. In some embodiments, a recombinant virus and/or plasmid used to generate a rAAV virus comprises other transcriptional or regulatory elements, such as a poly A (polyadenylation) sequence, untranslated regions (UTRs), 3′ UTRs, or termination sequences. In some embodiments, more than one gene is expressed from the vector or plasmid using internal ribosome entry site (IRES) or similar element that allows co-expression of two or more proteins or create multigene, or polycistronic mRNA.

In some embodiments, the rAAV and/or plasmid used to generate the rAAV comprises one or more of the following nucleic acid elements: a first ITR sequence; a promoter sequence; an intron sequence; a first UTR sequence; a heterologous nucleic acid encoding an anti-VEGF agent (e.g., aflibercept); a second UTR sequence; a polyA sequence; and a second ITR sequence. In some embodiments, linker sequence(s) are inserted between two or more of the nucleic acid elements. In some embodiments, the heterologous nucleic acid encoding a therapeutic polypeptide encodes aflibercept (or a functional fragment or functional variant thereof).

In some embodiments, a self-complementary vector (sc) can be used. The use of self-complementary AAV vectors may bypass the requirement for viral second-strand DNA synthesis and may lead to greater rate of expression of the transgene protein, as provided by Wu, Hum Gene Ther. 2007, 18(2):171-82, incorporated by reference herein.

In some aspects, several AAV vectors may be generated to allow selection of the most optimal serotype and promoter for use with the anti-VEGF agent transgene (e.g., aflibercept transgene).

In some embodiments, the AAV vector comprises a polynucleotide cassette for enhanced expression of a transgene (e.g., an anti-VEGF agent such as aflibercept) in a target cell (e.g., a retinal cell). In some embodiments, the polynucleotide cassette comprises in 5′ to 3′ order: (a) a first enhancer region comprising a CMV sequence (SEQ ID NO: 22); (b) a promoter region, comprising a CMV sequence (SEQ ID NO: 23); (c) a 5′UTR region comprising, in 5′ to 3′ order, TPL and eMLP sequences (SEQ ID NO: 24 and SEQ ID NO: 25, respectively); (d) a coding sequence encoding a peptide or polypeptide (e.g., an anti-VEGF agent such as aflibercept); (e) a second enhancer region comprising a full EES sequence (SEQ ID NO: 26); and (f) a HGH polyadenylation site (SEQ ID NO: 27). In certain of these embodiments, the polynucleotide cassette comprises one or more sequences selected from SEQ ID NO: 28-32 or a sequence with at least 85% identity thereto. In certain of these embodiments the 5′ arm of the polynucleotide cassette comprises or consists of SEQ ID NO: 33 or a sequence with at least 85% identity thereto. In certain of these embodiments the 3′ arm of the polynucleotide cassette comprises or consists of SEQ ID NO: 34 or a sequence with at least 85% identity thereto. The nucleic acid sequences of SEQ ID NOs: 22-34 are provided below:

(SEQ ID NO: 22)
ACTTACGGTA AATGGCCCGC CTGGCTGACC GCCCAACGAC
CCCCGCCCAT TGACGTCAAT AATGACGTAT GTTCCCATAG
TAACGCCAAT AGGGACTTTC CATTGACGTC AATGGGTGGA
GTATTTACGG TAAACTGCCC ACTTGGCAGT ACATCAAGTG
TATCATATGC CAAGTCCGCC CCCTATTGAC GTCAATGACG
GTAAATGGCC CGCCTGGCAT TATGCCCAGT ACATGACCTT
ACGGGACTTT CCTACTTGGC AGTACATCTA CGTATTAGTC
ATCGCTATTA CCA
(SEQ ID NO: 23)
TGCTGATGCG GTTTTGGCAG TACACCAATG GGCGTGGATA
GCGGTTTGAC TCACGGGGAT TTCCAAGTCT CCACCCCATT
GACGTCAATG GGAGTTTGTT TTGGCACCAA AATCAACGGG
ACTTTCCAAA ATGTCGTAAT AACCCCGCCC CGTTGACGCA
AATGGGCGGT AGGCGTGTAC GGTGGGAGGT CTATATAAGC
AGAGCTCGTT TAGTGAACCG
(SEQ ID NO: 24)
CTCACTCTCT TCCGCATCGC TGTCTGCGAG GGCCAGCTGT
TGGGCTCGCG GTTGAGGACA AACTCTTCGC GGTCTTTCCA
GTACTCTTGG ATCGGAAACC CGTCGGCCTC CGAACGGTAC
TCCGCCACCG AGGGACCTGA GCGAGTCCGC ATCGACCGGA
TCGGAAAACC TCTCGAGAAA GGCGTCTAAC CAGTCACAGT
CGCAAGGTAG GCTGAGCACC GTGGCGGGCG GCAGCGGGTG
GCGGTCGGGG TTGTTTCTGG CGGAGGTGCT GCTGATGATG
TAATTAAAGT AGGCGGTCTT GAGACGGCGG ATGGTCGA
(SEQ ID NO: 25)
CCAGCTGTTG GGGTGAGTAC TCCCTCTCAA AAGCGGGCAT
TACTTCTGCG CTAAGATTGT CAGTTTCCAA AAACGAGGAG
GATTTGATAT TCACCTGGCC CG
(SEQ ID NO: 26)
CTGTTCTCAT CACATCATAT CAAGGTTATA TACCATCAAT
ATTGCCACAG ATGTTACTTA GCCTTTTAAT ATTTCTCTAA
TTTAGTGTAT ATGCAATGAT AGTTCTCTGA TTTCTGAGAT
TGAGTTTCTC ATGTGTAATG ATTATTTAGA GTTTCTCTTT
CATCTGTTCA AATTTTTGTC TAGTTTTATT TTTTACTGAT
TTGTAAGACT TCTTTTTATA ATCTGCATAT TACAATTCTC
TTTACTGGGG TGTTGCAAAT ATTTTCTGTC ATTCTATGGC
CTGACTTTTC TTAATGGTTT TTTAATTTTA AAAATAAGTC
TTAATATTCA TGCAATCTAA TTAACAATCT TTTCTTTGTG
GTTAGGACTT TGAGTCATAA GAAATTTTTC TCTACACTGA
AGTCATGATG GCATGCTTCT ATATTATTTT CTAAAAGATT
TAAAGTTTTG CCTTCTCCAT TTAGACTTAT AATTCACTGG
AATTTTTTTG TGTGTATGGT ATGACATATG GGTTCCCTTT
TATTTTTTAC ATATAAATAT ATTTCCCTGT TTTTCTAAAA
AAGAAAAAGA TCATCATTTT CCCATTGTAA AATGCCATAT
TTTTTTCATA GGTCACTTAC ATATATCAAT GGGTCTGTTT
CTGAGCTCTA CTCTATTTTA TCAGCCTCAC TGTCTATCCC
CACACATCTC ATGCTTTGCT CTAAATCTTG ATATTTAGTG
GAACATTCTT TCCCATTTTG TTCTACAAGA ATATTTTTGT
TATTGTCTTT GGGCTTTCTA TATACATTTT GAAATGAGGT
TGACAAGTTA
(SEQ ID NO: 27)
CTGCCCGGGT GGCATCCCTG TGACCCCTCC CCAGTGCCTC
TCCTGGCCCT GGAAGTTGCC ACTCCAGTGC CCACCAGCCT
TGTCCTAATA AAATTAAGTT GCATCATTTT GTCTGACTAG
GTGTCCTTCT ATAATATTAT GGGGTGGAGG GGGGTGGTAT
GGAGCAAGGG GCCCAAGTTG GGAAGAAACC TGTAGGGCCT GC
(SEQ ID NO: 28)
AGGCGGTCTT GAGACGGCGG ATGGTCGAGG TGAGGTGTGG
CAGGCTTGAG ATCCAGCTGT TGGGGTGA
(SEQ ID NO: 29)
CGCTGTTTTG ACCTCCATAG TGGACACCGG GACCGATCCA
GCCTCCGCGT CTCAGGGGAG ATCTCGTTTA GTGAACCGTC
AGATCCTCAC TCTCTTCCGC ATCGCTGTCT GCGAGGGCCA
GCTGTTGGG
(SEQ ID NO: 30)
TTGATATTCA CCTGGCCCGA TCTGGCCATA CACTTG
(SEQ ID NO: 31)
CCCAGGTCCA AGTTTAAACG CC
(SEQ ID NO: 32)
TCTTTGGGCT TTCTATATAC ATTTTGAAAT GAGGTTGACA
AGTTACCTAG GAAAACTGTC TTCCTGCCCG GGTGGCA
(SEQ ID NO: 33)
CTCTGGAGAC GACTTACGGT AAATGGCCCG CCTGGCTGAC
CGCCCAACGA CCCCCGCCCA TTGACGTCAA TAATGACGTA
TGTTCCCATA GTAACGCCAA TAGGGACTTT CCATTGACGT
CAATGGGTGG AGTATTTACG GTAAACTGCC CACTTGGCAG
TACATCAAGT GTATCATATG CCAAGTCCGC CCCCTATTGA
CGTCAATGAC GGTAAATGGC CCGCCTGGCA TTATGCCCAG
TACATGACCT TACGGGACTT TCCTACTTGG CAGTACATCT
ACGTATTAGT CATCGCTATT ACCATGCTGA TGCGGTTTTG
GCAGTACACC AATGGGCGTG GATAGCGGTT TGACTCACGG
GGATTTCCAA GTCTCCACCC CATTGACGTC AATGGGAGTT
TGTTTTGGCA CCAAAATCAA CGGGACTTTC CAAAATGTCG
TAATAACCCC GCCCCGTTGA CGCAAATGGG CGGTAGGCGT
GTACGGTGGG AGGTCTATAT AAGCAGAGCT CGTTTAGTGA
ACCGTCAGAT CGCCTGGAGA GGCCATCCAC GCTGTTTTGA
CCTCCATAGT GGACACCGGG ACCGATCCAG CCTCCGCGTC
TCAGGGGAGA TCTCGTTTAG TGAACCGTCA GATCCTCACT
CTCTTCCGCA TCGCTGTCTG CGAGGGCCAG CTGTTGGGCT
CGCGGTTGAG GACAAACTCT TCGCGGTCTT TCCAGTACTC
TTGGATCGGA AACCCGTCGG CCTCCGAACG GTACTCCGCC
ACCGAGGGAC CTGAGCGAGT CCGCATCGAC CGGATCGGAA
AACCTCTCGA GAAAGGCGTC TAACCAGTCA CAGTCGCAAG
GTAGGCTGAG CACCGTGGCG GGCGGCAGCG GGTGGCGGTC
GGGGTTGTTT CTGGCGGAGG TGCTGCTGAT GATGTAATTA
AAGTAGGCGG TCTTGAGACG GCGGATGGTC GAGGTGAGGT
GTGGCAGGCT TGAGATCCAG CTGTTGGGGT GAGTACTCCC
TCTCAAAAGC GGGCATTACT TCTGCGCTAA GATTGTCAGT
TTCCAAAAAC GAGGAGGATT TGATATTCAC CTGGCCCGAT
CTGGCCATAC ACTTGAGTGA CAATGACATC CACTTTGCCT
TTCTCTCCAC AGGTGTCCAC TCCCAGGTCC AAGTTTAAAC
GCCGCCACCA TG
(SEQ ID NO: 34)
ACTGTTCTCA TCACATCATA TCAAGGTTAT ATACCATCAA
TATTGCCACA GATGTTACTT AGCCTTTTAA TATTTCTCTA
ATTTAGTGTA TATGCAATGA TAGTTCTCTG ATTTCTGAGA
TTGAGTTTCT CATGTGTAAT GATTATTTAG AGTTTCTCTT
TCATCTGTTC AAATTTTTGT CTAGTTTTAT TTTTTACTGA
TTTGTAAGAC TTCTTTTTAT AATCTGCATA TTACAATTCT
CTTTACTGGG GTGTTGCAAA TATTTTCTGT CATTCTATGG
CCTGACTTTT CTTAATGGTT TTTTAATTTT AAAAATAAGT
CTTAATATTC ATGCAATCTA ATTAACAATC TTTTCTTTGT
GGTTAGGACT TTGAGTCATA AGAAATTTTT CTCTACACTG
AAGTCATGAT GGCATGCTTC TATATTATTT TCTAAAAGAT
TTAAAGTTTT GCCTTCTCCA TTTAGACTTA TAATTCACTG
GAATTTTTTT GTGTGTATGG TATGACATAT GGGTTCCCTT
TTATTTTTTA CATATAAATA TATTTCCCTG TTTTTCTAAA
AAAGAAAAAG ATCATCATTT TCCCATTGTA AAATGCCATA
TTTTTTTCAT AGGTCACTTA CATATATCAA TGGGTCTGTT
TCTGAGCTCT ACTCTATTTT ATCAGCCTCA CTGTCTATCC
CCACACATCT CATGCTTTGC TCTAAATCTT GATATTTAGT
GGAACATTCT TTCCCATTTT GTTCTACAAG AATATTTTTG
TTATTGTCTT TGGGCTTTCT ATATACATTT TGAAATGAGG
TTGACAAGTT ACCTAGGAAA ACTGTCTTCC TGCCCGGGTG
GCATCCCTGT GACCCCTCCC CAGTGCCTCT CCTGGCCCTG
GAAGTTGCCA CTCCAGTGCC CACCAGCCTT GTCCTAATAA
AATTAAGTTG CATCATTTTG TCTGACTAGG TGTCCTTCTA
TAATATTATG GGGTGGAGGG GGGTGGTATG GAGCAAGGGG
CCCAAGTTGG GAAGAAACCT GTAGGGCCTG CGAAGACAGT CAG

Additional polynucleotide cassettes for enhanced expression of a transgene (e.g., a transgene encoding an anti-VEGF agent such as aflibercept) in a target cell (such as a retinal cell) are disclosed in WO 2018/170473, the contents of which are incorporated herein by reference in their entirety.

In some embodiments, the vector is a targeted vector, especially a targeted rAAV (e.g., AAV2.7m8) that shows higher infectivity of a specific cell, such as a retinal cell (e.g., a photoreceptor, a retinal ganglion cell, a Müller cell, a bipolar cell, an amacrine cell, a horizontal cell, or a retinal pigmented epithelium cell). Viral vectors for use in the disclosure can include those that exhibit low toxicity and/or low immunogenicity in a subject and expresses therapeutically effective quantities of the anti-VEGF agent (e.g., aflibercept) in a subject, e.g., human patient. Any suitable method known in the art can be used in the biochemical purification of recombinant viruses (e.g., rAAV), e.g., for the preparation of pharmaceutical compositions described elsewhere herein. Recombinant AAV viruses can be harvested directly from cells, or from the culture media comprising cells. Virus can be purified using various biochemical means, such as gel filtration, filtration, chromatography, affinity purification, gradient ultracentrifugation, or size exclusion methods. In some embodiments, the virus is lyophilized.

In some embodiments, the a rAAV comprises a 7m8 variant capsid protein, or rAAV2.7m8, and a nucleic acid sequence that encodes an anti-VEGF agent (e.g., aflibercept, or a functional fragment or functional variant thereof) in a subject (e.g., human or a non-human primate).

In some embodiments, the increase in retinal cell infectivity of rAAV variant (e.g., the 7m8 variant) is at least 5%, at least 10%, at least 20%, at least 30%, at least 40%, at least 50%, at least 60%, at least 70%, at least 80%, at least 90%, or at least 100% as compared to an AAV particle comprising the corresponding parental or unmodified AAV capsid protein. In some embodiments, the increase in infectivity of retinal cells is an increase of between 5% to 100%, between 5% to 95%, between 5% to 90%, between 5% to 85%, between 5% to 80%, between 5% to 75%, between 5% to 70%, between 5% to 65%, between 5% to 60%, between 5% to 55%, between 5% to 50%, between 5% to 45%, between 5% to 40%, between 5% to 35%, between 5% to 30%, between 5% to 25%, between 5% to 20%, between 5% to 15%, between 5% to 10% as compared to an AAV particle comprising the corresponding parental or unmodified AAV capsid protein.

In some embodiments, the increase in retinal cell infectivity of a rAAV variant is at least 1-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, or at least 2-fold compared to an AAV particle comprising the corresponding parental or unmodified AAV capsid protein. In some embodiments, the increase in infectivity is at least 2-fold, at least 3-fold, at least 4-fold, at least 5-fold, at least 6-fold, at least 7-fold, at least 8-fold, at least 9-fold, or at least 10-fold as compared to an AAV particle comprising the corresponding parental AAV capsid protein. In some embodiments, the increase in infectivity is at least 15-fold, at least 20-fold, at least 25-fold, at least 30-fold, at least 35-fold, at least 40-fold, at least 45-fold, at least 50-fold, at least 55-fold, at least 60-fold, at least 65-fold, at least 70-fold, at least 75-fold, at least 80-fold, at least 85-fold, at least 90-fold, or at least 100-fold compared to an AAV particle comprising the corresponding parental or unmodified AAV capsid protein.

In some embodiments, the increase in retinal cell infectivity is between 10-fold to 100-fold, between 10-fold to 95-fold, between 10-fold to 90-fold, between 10-fold to 85-fold, between 10-fold to 80-fold, between 10-fold to 75-fold, between 10-fold to 70-fold, between 10-fold to 65-fold, between 10-fold to 60-fold, between 10-fold to 55-fold, between 10-fold to 50-fold, between 10-fold to 45-fold, between 10-fold to 40-fold, between 10-fold to 35-fold, between 10-fold to 30-fold, between 10-fold to 25-fold, between 10-fold to 20-fold, or between 10-fold to 15-fold as compared to an AAV particle comprising the corresponding parental or unmodified AAV capsid protein.

In some embodiments, the increase in retinal cell infectivity is between 2-fold to 20-fold, between 2-fold to 19-fold, between 2-fold to 18-fold, between 2-fold to 17-fold, between 2-fold to 16-fold, between 2-fold to 15-fold, between 2-fold to 14-fold, between 2-fold to 13-fold, between 2-fold to 12-fold, between 2-fold to 11-fold, between 2-fold to 10-fold, between 2-fold to 9-fold, between 2-fold to 8-fold, between 2-fold to 7-fold, between 2-fold to 6-fold, between 2-fold to 5-fold, between 2-fold to 4-fold, or between 2-fold to 3-fold as compared to an AAV particle comprising the corresponding parental or unmodified AAV capsid protein.

In some embodiments, an amino acid modification of a capsid protein described herein can confer an increase in an ability to cross an internal limiting membrane (ILM) in an eye of a primate or human subject as compared to the ability of an AAV particle comprising the corresponding parental or unmodified AAV capsid protein to cross the ILM in the eye of the subject. In some embodiments, the increase in the ability to cross the ILM is an increase of at least 5%, at least 10%, at least 20%, at least 30%, at least 40%, at least 50%, at least 60%, at least 70%, at least 80%, at least 90%, or at least 100% as compared to an AAV particle comprising the corresponding parental or unmodified AAV capsid protein. In some embodiments, the increase in the ability to cross the ILM is an increase of between 5% to 100%, between 5% to 95%, between 5% to 90%, between 5% to 85%, between 5% to 80%, between 5% to 75%, between 5% to 70%, between 5% to 65%, between 5% to 60%, between 5% to 55%, between 5% to 50%, between 5% to 45%, between 5% to 40%, between 5% to 35%, between 5% to 30%, between 5% to 25%, between 5% to 20%, between 5% to 15%, or between 5% to 10% as compared to the parental or unmodified AAV capsid protein.

In some embodiments, the increase in the ability to cross the ILM is at least 1-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, or at least 2-fold compared to an AAV particle comprising the corresponding parental AAV capsid protein. In some embodiments, the increase in the ability to cross the ILM is at least 2-fold, at least 3-fold, at least 4-fold, at least 5-fold, at least 6-fold, at least 7-fold, at least 8-fold, at least 9-fold, or at least 10-fold as compared to an AAV particle comprising the corresponding parental AAV capsid protein. In some embodiments, the increase in the ability to cross the ILM is at least 15-fold, at least 20-fold, at least 25-fold, at least 30-fold, at least 35-fold, at least 40-fold, at least 45-fold, at least 50-fold, at least 55-fold, at least 60-fold, at least 65-fold, at least 70-fold, at least 75-fold, at least 80-fold, at least 85-fold, at least 90-fold, or at least 100-fold compared to an AAV particle comprising the corresponding parental or unmodified AAV capsid protein.

In some embodiments, the increase in the ability to cross the ILM is between 10-fold to 100-fold, between 10-fold to 95-fold, between 10-fold to 90-fold, between 10-fold to 85-fold, between 10-fold to 80-fold, between 10-fold to 75-fold, between 10-fold to 70-fold, between 10-fold to 65-fold, between 10-fold to 60-fold, between 10-fold to 55-fold, between 10-fold to 50-fold, between 10-fold to 45-fold, between 10-fold to 40-fold, between 10-fold to 35-fold, between 10-fold to 30-fold, between 10-fold to 25-fold, between 10-fold to 20-fold, or between 10-fold to 15-fold as compared to an AAV particle comprising the corresponding parental or unmodified AAV capsid protein.

In some embodiments, the increase in the ability to cross the ILM is between 2-fold to 20-fold, between 2-fold to 19-fold, between 2-fold to 18-fold, between 2-fold to 17-fold, between 2-fold to 16-fold, between 2-fold to 15-fold, between 2-fold to 14-fold, between 2-fold to 13-fold, between 2-fold to 12-fold, between 2-fold to 11-fold, between 2-fold to 10-fold, between 2-fold to 9-fold, between 2-fold to 8-fold, between 2-fold to 7-fold, between 2-fold to 6-fold, between 2-fold to 5-fold, between 2-fold to 4-fold, or between 2-fold to 3-fold as compared to an AAV particle comprising the corresponding parental or unmodified AAV capsid protein.

### Exemplary Anti-VEGF Agents for Delivery to Target Cells

In some embodiments, a gene therapy is used to deliver a transgene comprising a nucleic acid sequence that encodes or expresses an anti-VEGF agent when administered via intravitreal (IVT) injection to a subject (e.g., a human or a non-human primate). In some embodiments, rAAV comprising a capsid variant (e.g., AAV2.7m8) described herein comprises a heterologous nucleic acid sequence that encodes an anti-VEGF agent is used to deliver the sequence of the anti-VEGF agent gene into retinal cells upon intravitreal to a subject. In some embodiments, the rAAV comprising the gene encoding the anti-VEGF agent is formulated for gene therapy and intravitreal injection. In some embodiments, the gene encoding the anti-VEGF agent refers to a functional fragment or a functional variant thereof. In some embodiments, a “functional fragment” and/or a “functional variant” of an anti-VEGF agent refers to a fragment or variant of an anti-VEGF agent that is capable of producing a therapeutic effect when administered to the subject.

In some embodiments, the anti-VEGF agent is any therapeutic agent, including proteins, polypeptides, peptides, fusion protein, multimeric proteins, gene products, antibody, human monoclonal antibody, antibody fragment, aptamer, kinase inhibitor, receptor or receptor fragment, or nucleic acid molecule, that can reduce, interfere with, disrupt, block and/or inhibit the activity or function of an endogenous VEGF and/or an endogenous VEGF receptor (VEGFR), or the VEGF-VEGFR interaction or pathway in vivo. In some embodiments, the anti-VEGF agent is any one of the known therapeutic agents that can reduce new blood vessel growth or formation and/or edema, or swelling, when delivered into a cell, tissue, or a subject in vivo, e.g., ranibizumab, brolucizumab, or bevacizumab. In some embodiments, the anti-VEGF agent is naturally occurring, non-naturally occurring, or synthetic. In some embodiments, the anti-VEGF agent can be derived from a naturally occurring molecule that was subsequently modified or mutated to confer an anti-VEGF activity. In some embodiments, the anti-VEGF agent is a fusion or chimeric protein. In such proteins, functional domains or polypeptides are artificially fused to a moiety or a polypeptide to make a fusion or chimeric protein that can sequester VEGF in vivo or function as a VEGFR decoy. In some embodiments, the anti-VEGF agent is a fusion or chimeric protein that blocks endogenous VEGFR from interacting with its ligands.

In some embodiments, the anti-VEGF agent is bevacizumab. Bevacizumab (CAS Registry No. 216974-75-3; Drugbank Accession No. DB00112) is a recombinant humanized monoclonal IgG1 antibody that binds to all VEGF-A isoforms and blocks angiogenesis by inhibiting VEGF-A. Los, M.; Roodhart, J. M. L.; Voest, E. E. (2007). “Target Practice: Lessons from Phase III Trials with Bevacizumab and Vatalanib in the Treatment of Advanced Colorectal Cancer”. The Oncologist. 12 (4): 443-50; Shih, T; Lindley, C (November 2006). “Bevacizumab: an angiogenesis inhibitor for the treatment of solid malignancies.” Clinical therapeutics. 28 (11): 1779-802.

In some embodiments, the anti-VEGF agent is ranibizumab. Ranibizumab (CAS Registry No. 347396-82-1; DrugBank Accession No. DB01270) is a recombinant humanized IgG 1 kappa isotype monoclonal antibody fragment (Fab) and binds to all VEGF-A isoforms with a higher affinity than bevacizumab.

In some embodiments, the anti-VEGF agent is brolucizumab. Brolucizumab (CAS Registry No. 1531589-13-5) is a humanized single-chain antibody fragment (scFv) that binds all VEGF-A isoforms with high affinity.

In some embodiments, the anti-VEGF agent is aflibercept. The amino acid sequence of aflibercept is known in the art: C₄₃₁₈H₆₇₈₈N₁₁₆₄O₁₃₀₄S₃₂, FDA Unique Ingredient Identifier (UNIT) is 15C2VL427D. The amino acid sequence of aflibercept is available at DrugBank, accession number DB08885:

(SEQ ID NO: 35)
SDTGRPFVEM YSEIPEIIHM TEGRELVIPC RVTSPNITVT
LKKFPLDTLI PDGKRIIWDS RKGFIISNAT YKEIGLLTCE
ATVNGHLYKT NYLTHRQTNT IIDVVLSPSH GIELSVGEKL
VLNCTARTEL NVGIDFNWEY PSSKHQHKKL VNRDLKTQSG
SEMKKFLSTL TIDGVTRSDQ GLYTCAASSG LMTKKNSTFV
RVHEKDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP G

The nucleic acid sequence of aflibercept (SEQ ID NO: 36) is provided in FIG. 6.

As used herein, “aflibercept” refers to a polypeptide or protein sequence, or a functional fragment or variant or mutant thereof, with at least 75%, 80%, 81%, 82%, 83%, 84%, 85%, 86%, 87%, 88%, 89%, 90%, 91%, 92%, 93%, 94%, 95%, 96%, 97%, 98%, 99% or more, or 100% homology to the aflibercept amino acid sequence identified above. Homology refers to the % conservation of residues of an alignment between two sequences, including, but not limited to functional fragments, sequences comprising insertions, deletions, substitutions, pseudofragments, pseudogenes, splice variants or artificially optimized sequences.

In some embodiments, the amino acid sequence of aflibercept is at least 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% homologous to the aflibercept amino acid sequence of SEQ ID NO: 35. In some embodiments, the nucleic acid sequence used in a gene therapy or rAAV disclosed herein is compared to the corresponding cDNA sequence of the aflibercept amino acid sequence identified above, and shows at least 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% sequence homology between the nucleic acid sequences of aflibercept (e.g., SEQ ID NO: 36). In some embodiments, aflibercept is at least 80%, 85%, 90%, 91%, 92%, 93%, 94%, 95%, 96%, 97%, 98%, 99%, 99.9%, or 100% spatially homologous to aflibercept (e.g., in terms of its secondary, tertiary, and quaternary structure or conformation). In some embodiments, aflibercept of the pharmaceutical compositions and methods disclosed herein is at most 80%, 85%, 90%, 91%, 92%, 93%, 94%, 95%, 96%, 97%, 98%, 99%, 99.9%, or 100% spatially homologous to the aflibercept used in the standard of care (e.g., secondary, tertiary, and quaternary structure or conformation).

In some embodiments, the aflibercept gene product, or aflibercept transgene, as included in a gene therapy based on a rAAV comprises a capsid variant as disclosed herein (e.g., the 7m8 variant), encodes a protein, fusion protein, or polypeptide that has at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% homology to the above amino acid sequence of SEQ ID NO: 35, or between the corresponding cDNA sequences of aflibercept (e.g., cDNA of aflibercept sequence used in a gene therapy compared to SEQ ID NO: 36). In some embodiments, methods and pharmaceutical compositions disclosed herein comprise a functional fragment of aflibercept, or a variant or mutant thereof. In some embodiments, the nucleic acid sequence of aflibercept is modified or codon-optimized to enhance its activity, expression, stability, and/or solubility in vivo.

In some embodiments, the nucleic acid sequence of aflibercept is derived from its amino acid sequence. In some embodiments, the nucleic acid sequence of aflibercept is codon optimized to improve its expression in a subject.

Codon optimization can be achieved with any method known in the art. Codon optimization refers to a process of modifying a nucleic acid sequence for enhanced expression of a gene in target or host cells of interest, e.g., human retinal cells, by replacing at least one codon (e.g., about or more than 1, 2, 3, 4, 5, 10, 15, 20, 25, 50, 100 or more codons) of a native sequence with codons that are used more frequently or are most frequently used in the host cell while maintaining the native amino acid sequence. Codon usage tables are readily available, including for examples, GenScript Codon Usage Frequency Table Tool at www(dot)genscript(dot)com/tools/codon-frequency-table; Codon Usage Database at www(dot)kazusa(dot)or(dot)jp/codon/; and Nakamura, Y., et al. “Codon usage tabulated from the international DNA sequence databases: status for the year 2000” Nucl. Acids Res. 28:292 (2000).

Aflibercept is a 115 kDa fusion protein, which can be glycosylated. Aflibercept comprises an IgG backbone fused to extracellular VEGF receptor sequences of the human VEGFR-1 and VEGFR-2, and functions like a soluble decoy receptor by binding VEGF-A with a greater affinity than its natural or endogenous receptors. See, for example, Stewart MW. Aflibercept (VEGF Trap-eye): the newest anti-VEGF drug. Br. J. Ophthalmol. 2012 September; 96(9):1157-8. Aflibercept's high affinity for VEGF interferes or disrupts subsequent binding and activation of native or endogenous VEGF receptors. Reduced VEGF activity can lead to decreased angiogenesis and vascular permeability. Inhibition of placental growth factor PIGF and VEGF-B by aflibercept may also contribute to the treatment of ocular diseases or disorders characterized by abnormal (e.g., excessive) angiogenesis and/or neovascularization. PIGF has been associated with angiogenesis and certain ocular diseases or disorders, such as wet AMD, may be associated with elevated levels of PIGF. VEGF-B overexpression can be associated with breakdown of the blood-retinal barrier and retinal angiogenesis. Thus, inhibition of VEGF-A, VEGF-B, and PIGF may all contribute to the efficacy of aflibercept.

In some embodiments, the nucleic acid sequence of aflibercept is codon-optimized for expression in a primate or a human subject. Construction of a synthetic gene corresponding to the aflibercept amino acid sequence has been described in literature, e.g., Kanda A, Noda K, Saito W, Ishida S. Aflibercept Traps Galectin-1, an Angiogenic Factor Associated with Diabetic Retinopathy. Scientific Reports 5:17946 (2015) (describing “VEGF-Trap_{R1R2} (corresponding to aflibercept) cDNA was generated as a synthetic gene by IDT (Coralville, Iowa)”). Given the available amino acid sequence of aflibercept, any method known in the art can be used to generate the cDNA of aflibercept for use in a gene therapy or a rAAV described herein.

In some embodiments, AAV2.7m8 is used as a gene therapy or delivery system for aflibercept. AAV2.7m8-aflibercept refers to a rAAV2 comprising the 7m8 insertion between positions 587 and 588 in capsid protein VP1 of rAAV2 and a nucleic acid sequence encoding aflibercept.

### Ocular Diseases or Disorders

In some embodiments, the methods provided herein are suitable for use in the treatment of ocular diseases or disorders that arise asynchronously in each eye. Examples of diseases that may arise asynchronously (arising first in one eye, and later or not at all in the second eye) include glaucoma, wAMD, RVO, DME, DR and others. In some embodiments, the methods provided herein are suitable for use in the treatment of ocular diseases or disorders where simultaneous treatment of both eyes is unfeasible, inadvisable, and/or unsafe. In some embodiments, an rAAV particle of any serotype comprising the 7m8 variant (e.g., rAAV2.7m8) or a pharmaceutical composition thereof as described herein is used to treat or at least partially ameliorate an ocular disease or disorder associated with abnormal (e.g., excessive) neovascularization of the eye, e.g., wherein abnormal (e.g., excessive) neovascularization arises asynchronously in each eye. In some embodiments, a rAAV particle comprising a capsid variant protein is used to deliver an anti-VEGF agent (e.g., aflibercept, a functional fragment, or variant thereof) into an eye of a human subject.

Ocular disease or disorders that are approved for treatment with an anti-VEGF agent (e.g., bevacizumab, brolucizumab, ranibizumab, aflibercept, etc.) include, e.g., neovascular (wet) age-related macular degeneration (wAMD), macular edema following retinal vein occlusion (RVO), diabetic macular edema (DME) and diabetic retinopathy (DR) in patients with DME. In some embodiments, methods and pharmaceutical compositions disclosed herein are used to prevent or treat an ocular disease or disorder for which bevacizumab, brolucizumab, ranibizumab, and/or aflibercept is approved or indicated. In some embodiments, a gene therapy (e.g., AAV2.7m8 based gene therapy) is used to treat or prevent an ocular disease or disorder that is responsive to bevacizumab, brolucizumab, ranibizumab, and/or aflibercept, including, but not limited to, CNV, wet AMD, dry AMD, DME, RVO, macular edema following RVO, and diabetic retinopathy in patients with DME. In some embodiments, a rAAV gene therapy is used to treat or prevent any ocular disease or disorder characterized by neovascularization or CNV. In some embodiment, the methods and kits provided herein are for the treatment of diseases such as AMD, DME, RVO, angiogenesis related diseases, cancer, autoimmune diseases, infectious disease organisms, and the like.

In some embodiments, the ocular disease or disorder treated according to the methods described herein is diabetic macular edema. Diabetic macular edema (DME) is a swelling of the retina in diabetes mellitus due to leaking of fluid from blood vessels within the macula. The macula is the central portion of the retina, a small area rich in cones, the specialized nerve endings that detect color and upon which daytime vision depends. As macular edema develops, blurring occurs in the middle or just to the side of the central visual field. Visual loss from diabetic macular edema can progress over a period of months and make it impossible to focus clearly. Common symptoms of DME are blurry vision, floaters, double vision, and eventually blindness if it goes untreated. In some embodiments, methods and pharmaceutical compositions as disclosed herein are used to treat DME.

In some embodiments, the ocular disease or disorder treated according to the methods described herein is a retinal vein occlusion. Retinal vein occlusion is a blockage of the small veins that carry blood away from the retina. The retina is the layer of tissue at the back of the inner eye that converts light images to nerve signals and sends them to the brain. Retinal vein occlusion is most often caused by hardening of the arteries (atherosclerosis) and the formation of a blood clot. Blockage of smaller veins (branch veins or BRVO) in the retina often occurs in places where retinal arteries that have been thickened or hardened by atherosclerosis cross over and place pressure on a retinal vein. Symptoms of retinal vein occlusion can include a sudden blurring or vision loss in all or part of one eye.

In some embodiments, the ocular disease or disorder treated according to the methods described herein is choroidal neovascularization (CNV), also known as wet age-related macular degeneration (wAMD). Choroidal neovascularization can involve the growth of new blood vessels that originate from the choroid through a break in the Bruch membrane into the sub-retinal pigment epithelium (sub-RPE) or subretinal space, which can be a major cause of visual loss. CNV can create a sudden deterioration of central vision, noticeable within a few weeks. Other symptoms can include color disturbances, and metamorphopsia (distortions in which straight lines appears wavy). Hemorrhaging of the new blood vessels can accelerate the onset of symptoms of CNV. CNV may also include feeling of pressure behind the eye.

The advanced “wet” form (neovascular or exudative) of AMD is less common, but may frequently cause a rapid and often substantial loss of central vision in patients. In the wet form of AMD, choroidal neovascularization forms and develops into a network of vessels that may grow under and through the retinal pigment epithelium. As this is accompanied by leakage of plasma and/or hemorrhage into the subretinal space, there could be severe sudden loss of central vision if this occurs in the macula. The term “AMD”, if not otherwise specified, can be either dry AMD or wet AMD. The present disclosure contemplates treatment or prevention of AMD, wet AMD and/or dry AMD. In some embodiments, methods and pharmaceutical compositions as disclosed herein are used to treat AMD.

In some embodiments, methods described herein are used to prevent or treat an ocular disease or disorder that is responsive to treatment with bevacizumab, brolucizumab, ranibizumab, and/or aflibercept. In some embodiments, methods described herein are used to prevent or treat an ocular disease or disorder in a subject who has received prior treatment with bevacizumab, brolucizumab, ranibizumab, and/or aflibercept.

In some embodiments, methods and pharmaceutical compositions disclosed herein, i.e., AAV gene therapy comprising an anti-VEGF agent (e.g., bevacizumab, brolucizumab, ranibizumab, and/or aflibercept, or a functional fragment or variant thereof), results in a reduction in neovascularization or CNV, as measured by percentage of grade IV lesions following CNV formation according to color fundus photography, by at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% as compared to a vehicle or buffer control.

In some embodiments, methods and pharmaceutical compositions disclosed herein, i.e., AAV gene therapy comprising an anti-VEGF agent (e.g., bevacizumab, brolucizumab, ranibizumab, and/or aflibercept, or a functional fragment or variant thereof) results in a reduction in neovascularization or CNV, as measured by percentage of grade IV lesions following CNV formation according to color fundus photography, that is comparable to, e.g., an anti-VEGF agent (e.g., bevacizumab, brolucizumab, ranibizumab, and/or aflibercept, or a functional fragment or variant thereof) or a non-gene therapy-based anti-VEGF agent. In some embodiments, the reduction in CNV, or the therapeutic effect, lasts longer with the administration of a gene therapy comprising an anti-VEGF agent (e.g., bevacizumab, brolucizumab, ranibizumab, and/or aflibercept, or a functional fragment or variant thereof) as compared to administration with a non-gene therapy-based anti-VEGF agent (e.g., bevacizumab, brolucizumab, ranibizumab, and/or aflibercept, or a functional fragment or variant thereof) or a protein solution of the anti-VEGF agent (e.g., bevacizumab, brolucizumab, ranibizumab, and/or aflibercept, or a functional fragment or variant thereof). In some embodiments, the therapeutic effect of anti-VEGF gene therapy (e.g., bevacizumab, brolucizumab, ranibizumab, and/or aflibercept, or a functional fragment or variant thereof) lasts for at least 1 year, 1.5, 2, 3, 4, 5, 6, 7, 8, 9, 10 or more years after a single intravitreal injection. In some embodiments, pharmaceutical compositions disclosed herein inhibit or sequester endogenous VEGF and/or PIGF.

### Pharmaceutical Compositions

Provided herein are pharmaceutical compositions (such as pharmaceutical formulations) comprising one or more active ingredients, e.g., an AAV2.7m8 vector that comprises a nucleic acid sequence that encodes the anti-VEGF agent (e.g., bevacizumab, brolucizumab, ranibizumab, and/or aflibercept, or a functional fragment or variant thereof) as well as one or more excipients, carriers, stabilizers, or bulking agents. The pharmaceutical compositions are suitable for administration to a human patient via intravitreal (IVT) injection to achieve a desired therapeutic or prophylactic effect.

In some embodiments, the pharmaceutical composition comprising, e.g., an AAV2.7m8 vector that comprises a nucleic acid sequence that encodes the anti-VEGF agent (e.g., bevacizumab, brolucizumab, ranibizumab, and/or aflibercept, or a functional fragment or variant thereof), is supplied as a reconstituted homogenous solution. In some embodiments, the solution is a suspension. In some embodiments, the solution is isotonic. In other embodiments, the pharmaceutical composition comprising e.g., an AAV2.7m8 vector that comprises a nucleic acid sequence that encodes the anti-VEGF agent (e.g., bevacizumab, brolucizumab, ranibizumab, and/or aflibercept, or a functional fragment or variant thereof), is supplied in a lyophilized form, and is reconstituted prior to administration to a patient. In some embodiments, the methods provided herein further comprise the steps of reconstituting, dissolving, or solubilizing a lyophilized pharmaceutical composition comprising rAAV (e.g., AAV2.7m8) and encodes anti-VEGF agent (e.g., bevacizumab, brolucizumab, ranibizumab, and/or aflibercept, or a functional fragment or variant thereof) in a buffer prior to administration to the subject. In some embodiments, such lyophilized pharmaceutical composition comprises one or more of the following: a cryoprotectant, a surfactant, a salt, a stabilizer, or any combination thereof.

In some embodiments, the pharmaceutical composition is a homogenous solution. In some embodiments, the homogenous solution is supplied in a pre-filled syringe. In some embodiments, the pharmaceutical composition is supplied as a suspension. In some embodiments, a suspension is a solution. In some embodiments, the suspension is refrigerated. In some embodiments, method provided herein further comprise the step of warming the refrigerated suspension to room temperature and/or agitating the suspension to ensure that the active ingredient(s) are dissolved and/or evenly distributed in solution prior to administering the pharmaceutical to the subject (e.g., via IVT injection). In some embodiments, the suspension is diluted prior to administration to the subject (e.g., via IVT injection). In some embodiments, the suspension is supplied as a pre-filled syringe.

In some embodiments, the pharmaceutical composition is provided as a refrigerated suspension. In some embodiments, the suspension comprises a pharmaceutically acceptable excipient, e.g., surfactant, glycerol, non-ionic surfactant, buffer, glycol, salt, and any combination thereof. In some embodiments, hydrochloric acid and sodium hydroxide are used to adjust the pH of the solution. In some embodiments, the refrigerated suspension is at a neutral pH, or at a pH between about 6.5 and about 7.5. In some embodiments, the pH of the refrigerated suspension is slightly basic (e.g., having a pH of about any one of 7.5, 8, 8.2, 8.4, 8.5, or 9, including any range in between these values). In some embodiments, the pH of the suspension or solution is slightly acidic (e.g., having a pH of about 6.5, 6.3, 6.1, 6, 5.5, or 5, including any range in between these values). In some embodiments, the suspension is a solution. In some embodiments, the suspension comprises micelles. In some embodiments, suspension is agitated and/or warmed to room temperature before administration to the subject (e.g., via IVT injection).

Also provided herein are kits comprising at least one pharmaceutical composition described herein. In some embodiments, the kit comprises a comprising lyophilized or freeze-dried pharmaceutical composition (e.g., one unit dose in a vial) disclosed herein and a solution for dissolving, diluting, and/or reconstituting the lyophilized pharmaceutical composition. In some embodiments, the solution for reconstituting or dilution is supplied as a pre-filled syringe. In some embodiments, a kit comprises a freeze-dried or lyophilized pharmaceutical composition comprising rAAV (e.g., AAV2.7m8) and a solution for reconstituting the pharmaceutical composition to a desired concentration or volume. In some embodiments, the kit includes a buffer that helps to prevent aggregation upon reconstituting the pharmaceutical composition disclosed herein. In some embodiments, the pharmaceutical composition is provided in a pre-filled syringe. In some embodiments, a kit comprises a dual-chamber syringe or container wherein one of the chambers contains a buffer for dissolving or diluting the pharmaceutical composition. In some embodiments, the kit comprises a syringe for injection. In some embodiments, the reconstituted solution is filtered before administration. In some embodiments, the kit comprises a filter or a filter syringe for filtering the reconstituted pharmaceutical composition before administration to a patient.

In some embodiments, for storage stability and convenience of handling, a pharmaceutical composition, comprising rAAV (e.g., AAV2.7m8) and a nucleic acid sequence that encodes the anti-VEGF agent (e.g., bevacizumab, brolucizumab, ranibizumab, and/or aflibercept, or a functional fragment or variant thereof), is formulated as a lyophilized, freeze dried, or vacuum dried powder that is reconstituted with saline, buffer, or water prior to administration to a subject. Alternately, the pharmaceutical composition is formulated as an aqueous solution, such as a suspension or a homogeneous solution. A pharmaceutical composition can contain rAAV particles comprising a nucleic acid sequence that encodes aflibercept. In some embodiments, a different virus or delivery system, e.g., nanoparticles or lipid-based complexes, is used to deliver the nucleic acid sequence that encodes the anti-VEGF agent (e.g., bevacizumab, brolucizumab, ranibizumab, and/or aflibercept, or a functional fragment or variant thereof). Various excipients, such as phosphate, PBS, or Tris buffer, glycol, glycerol, saline, surfactant (e.g., pluronic or polysorbate), or any combination thereof, can be used to stabilize a pharmaceutical composition. Additionally, cryoprotectants, such as alcohols can be used as a stabilizer under freezing or drying conditions of lyophilization. In some embodiments, the gene therapy is provided as a suspension or a refrigerated suspension.

In some embodiments, a suspension or a reconstituted form of the lyophilized pharmaceutical composition comprising the anti-VEGF agent (e.g., aflibercept) gene therapy as disclosed herein has a volume of about 10, 20, 30, 40, 50, 60, 70, 80, 90, 100, 200, 300, 400, 500, 600, 700, 800, 900, or 1000 μL. In some embodiments, the suspension of the pharmaceutical composition comprising the anti-VEGF agent (e.g., aflibercept) gene therapy as disclosed herein has a volume of between 0.1 to 0.5 mL, between 0.1 to 0.2 mL, between 0.3 to 0.5 mL, between 0.5-1.0 mL, between 0.5-0.7 mL, between 0.6 to 0.8 mL, between 0.8 to 1 mL, between 0.9 to 1.1 mL, between 1.0 to 1.2 mL, or between 1.0 to 1.5 mL. In other embodiments, the volume is no more than 0.1, 0.2, 0.3, 0.4, 0.5, 0.6, 0.7, 0.8, 0.9, 1.0, 1.1, 1.2, 1.3, 1.4, or 1.5 mL.

In some embodiments, pharmaceutical compositions disclosed herein are designed, engineered, or adapted for administration to a primate (e.g., non-human primate and human subjects) via intravitreal or subretinal injection. In some embodiments, a pharmaceutical composition comprising rAAV particles comprising a nucleic acid sequence that encodes the anti-VEGF agent (e.g., aflibercept) is formulated for intravitreal injection into an eye of a subject. In some embodiments, the pharmaceutical composition is formulated to or reconstituted to a concentration that allows intravitreal injection of a volume not more than about or not more than 2, 2.5, 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μL. In some embodiments, a unit dose of the pharmaceutical composition comprises a volume not more than about or not more than 2, 2.5, 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μL. In some embodiments, methods of treatment disclosed herein comprises intravitreal injection of a volume of about 2, 5, 10, 15, 20, 25, 30, 35, 40, 45, 50, 55, 60, 65, 70, 75, 80, 85, 90, 95, 100, 110, 120, 130, 140, 150 μL of a solution comprising a rAAV (e.g., AAV2.7m8) and a nucleic acid sequence that encodes the anti-VEGF agent (e.g., aflibercept).

In some embodiments, an AAV2.7m8 particle comprising a nucleic acid sequence of the anti-VEGF agent (e.g., aflibercept) transgene described herein is a component of a gene therapy pharmaceutical composition. In some embodiments, a rAAV particle of any serotype comprising the 7m8 variant capsid protein as described herein is used to make a freeze-dried or lyophilized pharmaceutical composition or a suspension thereof. In some embodiments, the gene therapy is formulated as a refrigerated suspension. In some embodiments, the rAAV particle is rAAV2. In some embodiments, the lyophilized or suspension of the pharmaceutical composition comprises rAAV2 having the 7m8 variant capsid protein and a DNA sequence that encodes the anti-VEGF agent (e.g., aflibercept). In some embodiments, the suspension is refrigerated.

In some embodiments, the pharmaceutical composition is a unit dose (e.g., a therapeutically effective dose) to be administered to a subject (e.g., a human or non-human primate) via IVT injection for the treatment of an ocular disease or disorder characterized by abnormal (e.g., excessive) angiogenesis or neovascularization. In some embodiments, the pharmaceutical composition comprises a unit dose (e.g., a therapeutically effective dose) as described in further detail elsewhere herein. In some embodiments, the volume of the unit dose (e.g., a therapeutically effective dose) of a viral vector (e.g., an rAAV vector disclosed herein) administered to the subject is no more than about any one of about 50, 40, 30, 20, 10, or 5 μL, including any range in between these values. Minimizing the volume of the unit dose to be administered to the subject may obviate or mitigate changes in ocular pressure and other adverse effects associated with IVT injection (e.g., elevated intraocular pressure, inflammation, irritation, or pain).

Pharmaceutical compositions suitable for ocular use include sterile aqueous solutions or dispersions and sterile powders for the extemporaneous preparation of sterile injectable solutions, suspension, or dispersion. For intravitreal administration, suitable carriers include physiological saline, bacteriostatic water, phosphate buffered saline (PBS), and/or an isotonic agent, e.g., glycerol. In all embodiments, the pharmaceutical composition must be sterile and should be fluid to the extent that easy syringeability or injectability exists. It must be stable under the conditions of manufacture and storage and must be preserved against the contaminating action of microorganisms such as bacteria and fungi. In some embodiments, the pharmaceutical composition can include an isotonic agent, such as a salt or glycerol. In some embodiments, a surfactant or a stabilizer is added to the pharmaceutical composition to prevent aggregation.

In some embodiments, the excipient is a carrier. A carrier is a solvent or dispersion medium containing, for example, water, saline, ethanol, a polyol (for example, glycerol, propylene glycol, and liquid polyethylene glycol, and the like), and any combination thereof. The proper fluidity can be maintained, for example, by the use of a coating such as lecithin, by the maintenance of the required particle size in the case of dispersion and by the use of surfactants such as polysorbates (e.g., Tween™, polysorbate 20, polysorbate 80), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol (Triton X100™), N,N-dimethyldodecylamine-N-oxide, hexadecyltrimethylammonium bromide (HTAB), polyoxyl 10 lauryl ether, Brij 721™, bile salts (sodium deoxycholate, sodium cholate), pluronic acids (F-68, F-127), polyoxyl castor oil (Cremophor™) nonylphenol ethoxylate (Tergitol™), cyclodextrins and, ethylbenzethonium chloride (Hyamine™) Prevention of the action of microorganisms can be achieved by various antibacterial and antifungal agents, for example, parabens, chlorobutanol, phenol, ascorbic acid, cresol, thimerosal, and the like. In many embodiments, it will be preferable to include isotonic agents, for example, sugars, poly alcohols such as mannitol, sorbitol, sodium chloride in the composition. Prolonged absorption of the internal compositions can be brought about by including in the composition an agent which delays absorption, for example, aluminum monostearate and gelatin. In some embodiments, the pharmaceutical carrier includes sodium phosphate, sodium chloride, polysorbate, and sucrose. In some embodiments, a pharmaceutical composition comprises a surfactant, e.g., non-ionic surfactant such as polysorbate, poloxamer, or pluronic. In some embodiments, the addition of a non-ionic surfactant reduces aggregation in the pharmaceutical composition.

### Articles of Manufacture and Kits

In some embodiments, provided are kits that comprise one or more pharmaceutical compositions disclosed herein for use according to a method described herein. In some embodiments, the kit comprises a recombinant viral vector (e.g., rAAV or rAAV2.7m8 comprising the nucleic acid sequence of the anti-VEGF agent (e.g., aflibercept)). In some aspects, the kit comprises a lyophilized form of a pharmaceutical composition and a solution for reconstituting the pharmaceutical composition prior to administration to a subject. In some embodiments, a kit comprises: a recombinant virus provided herein, and instructions to administer a unit dose of a pharmaceutical composition to a first eye the subject via intravitreal (IVT) injection at a first time point, and to administer a second unit dose of the pharmaceutical composition to a contralateral eye of the subject via IVT injection at a second time point, according to any one of the methods described elsewhere herein.

In some embodiments, the kit comprises pharmaceutically acceptable excipients, buffers, solutions, etc. for administering the pharmaceutical composition. In some embodiments, the kit further comprises instructions for suitable operational parameters in the form of a label or a separate insert. For example, the kit may have standard instructions informing a physician or laboratory technician to prepare a unit dose (e.g., a therapeutically effective dose) of the pharmaceutical composition and/or to reconstitute lyophilized compositions. In some embodiments, the kit further comprises a device for administration, such as a syringe, filter needle, extension tubing, cannula, or other implements to facilitate intravitreal injection of the pharmaceutical composition.

In some embodiments, the kit comprises a pharmaceutical composition in the form of a suspension or refrigerated suspension, and a syringe and/or a buffer for dilution. In some embodiments, the kit comprises a pre-filled syringe comprising the suspension or refrigerated suspension.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: Sequential Intravitreal Administration of AAV2.7m8-Aflibercept Gene Therapy to the Contralateral Eyes in Non-Human Primates

The ability of AAV vectors (e.g., AAV2.7m8) to efficiently transduce target retinal cells has been exploited to successfully transfer therapeutic genes into photoreceptors, retinal pigment epithelium, and the inner retina to treat a variety of retinal diseases. Intravitreal (IVT) AAV administration is a safe and convenient method of retinal delivery, but it has been suggested that neutralizing antibodies (nAb) against the vector capsid are more likely to be generated following IVT than following subretinal injection. Given that certain ocular diseases, such as wet age-related macular degeneration (wAMD), can affect both of an individual's eyes, there is a concern that nAb generated following IVT administration of an AAV to a first eye may decrease the efficiency of therapeutic gene transfer and prevent effective vector re-administration, e.g., to the individual's contralateral eye.

The experiments described below were conducted to assess the effect of prior exposure of AAV2.7m8-aflibercept in one eye on transduction efficacy of the same AAV vector in the contralateral eye of non-human primates (i.e., St. Kitts African green monkeys). AAV2.7m8-aflibercept is a recombinant, replication-deficient adeno-associated virus (AAV.7m8) vector carrying a coding sequence for aflibercept.

Briefly, four monkeys were selected for the study. Prior to the beginning of the study, each monkey was examined and determined to have normal slit lamp and fundus exams, color fundus photographs (CFP), and optical coherence tomography (OCT). Additionally, each monkey was found to be negative for AAV.7m8 neutralizing antibodies (nAb) titers in an in vitro HEK293T cell-based assay prior to the study. On Day 0, three monkeys were administered with 2E12vg AAV2.7m8-aflibercept to the right eye (i.e., OD) via intravitreal injection (IVT), and one monkey was administered with vehicle to both eyes (i.e., OU). The volume of a human eye is approximately two times that of the African green monkey. Therefore, the dose administered to the monkeys (2E12 vg/eye) is equivalent to 4E12 vg/eye on a volume:volume basis. On Day 59, the three monkeys that had received AAV2.7m8-aflibercept OD were IVT administered with 2E12vg AAV2.7m8-aflibercept to the left eye (i.e., OS). See Table A below.

TABLE A
Staggered Dosing of AAV2.7m8-Aflibercept in Contralateral Eyes

| | | | | | Follow up |
|---|---|---|---|---|---|
| | N | | Treatment | Eye | post- |
| Treatment | (number) | Dose (IVT) | delivery | Treated | treatment |
| AAV2.7m8- | 3 | 2 × 10¹² vg/ | Day 0 | OD | 264 Days |
| aflibercept | monkeys | 100 μL | | | (9 months) |
| | | 2 × 10¹² vg/ | Day 59 | OS | 205 Days |
| | | 100 μL | | | (7 months) |
| Vehicle | 1 | 100 μL | Day 0 | OU | 264 Days |
| | monkey | | | | (9 months) |

Vitreous fluid and aqueous fluid samples were obtained from each of the four monkeys on Days 28, 56, 84, 112, 140, 168, 196, and 224 of the study and assessed via ELISA for aflibercept expression. Serum samples were collected in parallel and evaluated for the presence of neutralizing antibodies (nAb) that react with the 7m8 capsid using an in vitro HEK293T cell-based assay. Baseline samples of vitreous fluid and plasma were obtained prior to the study. Aflibercept expression in various ocular tissues was determined via ELISA post-mortem at study termination (i.e., Day 264).

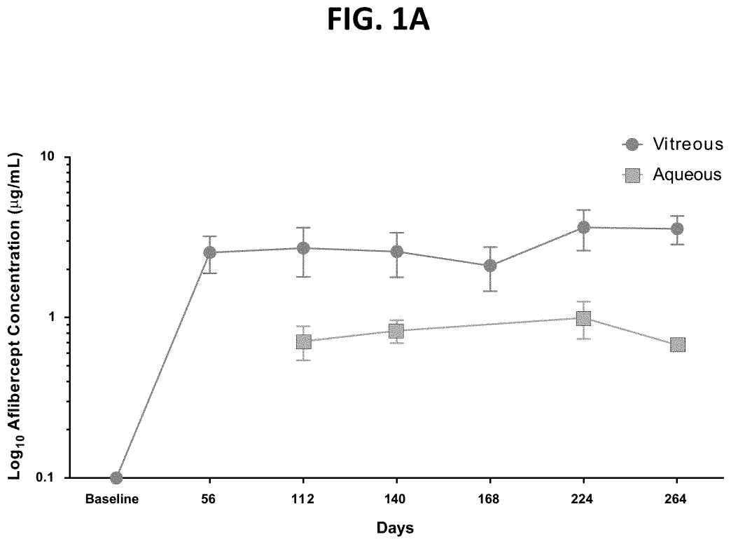

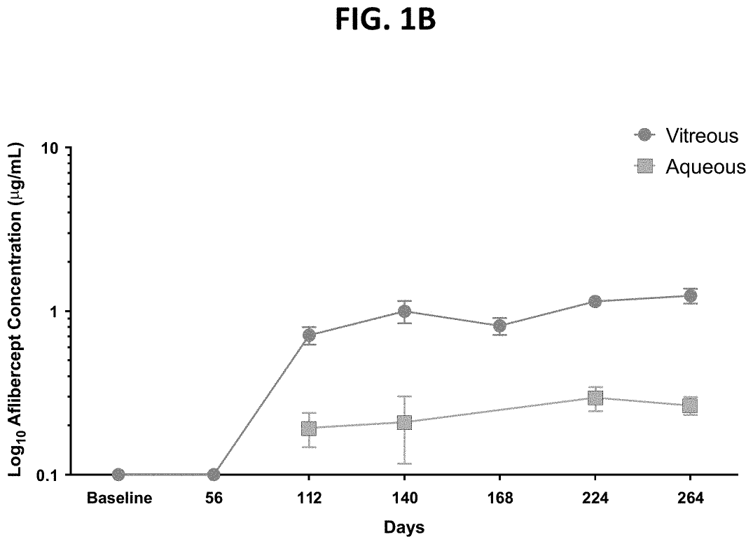

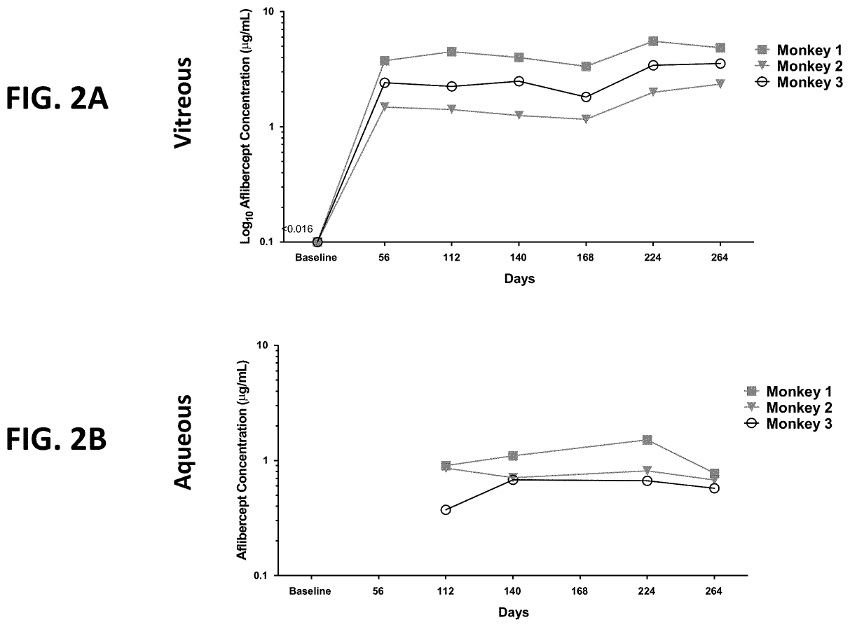

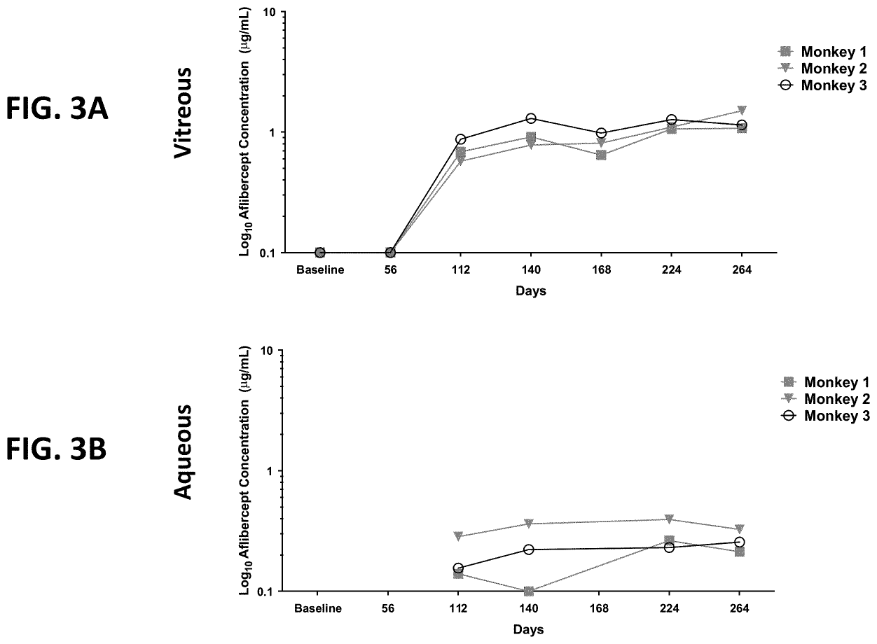

A time course of average aflibercept expression in the vitreous and aqueous fluids of the right eyes of the three monkeys that received AAV2.7m8-aflibercept on Day 0 of the study is shown in FIG. 1A. The expression levels of aflibercept are consistent with historical data for the same dose. FIG. 1B shows a time course of average aflibercept expression in the vitreous and aqueous fluids of the left eyes (i.e., the later dosed eyes) of the three monkeys that received AAV2.7m8-aflibercept on Day 59 of the study. Aflibercept expression levels in the left eyes (which received the later dose of AAV2.7m8-aflibercept) were about 3-fold lower than the expression levels in the right eyes at equivalent time points following IVT. Such expression level in the left eye is within the range that has been shown to provide therapeutic levels of aflibercept in a non-human primate model of choroidal neovascularization. FIG. 1A represents the average of the three expression levels shown in FIGS. 2A (vitreous) and 2B (aqueous). FIG. 1B represents the average of the three expression levels shown in FIGS. 3A (vitreous) and 3B (aqueous).

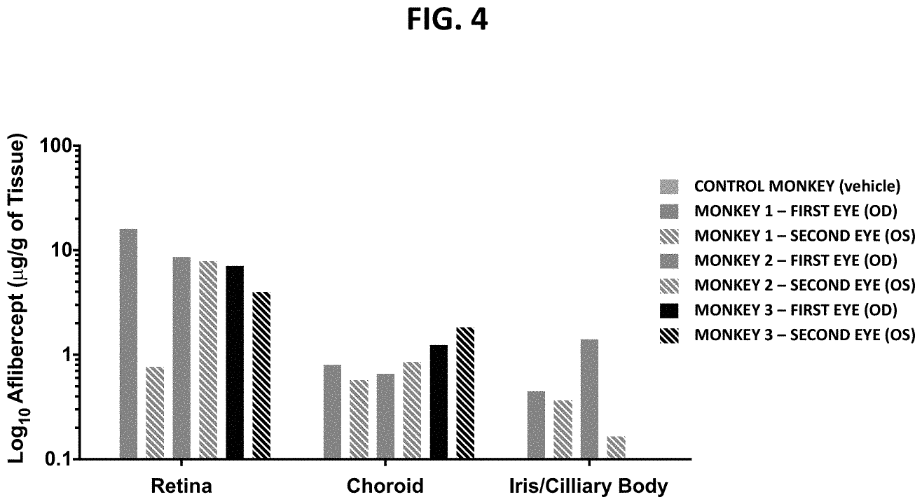

FIG. 4 shows expression levels of aflibercept in the retina, choroid, and iris/ciliary body in each eye of each monkey at study termination (i.e., on Day 264). In general, expression of aflibercept was highest in the retina. Aflibercept expression was found to be higher in the first eyes (i.e., OD) of each monkey that was administered with AAV2.7m8-aflibercept via IVT.

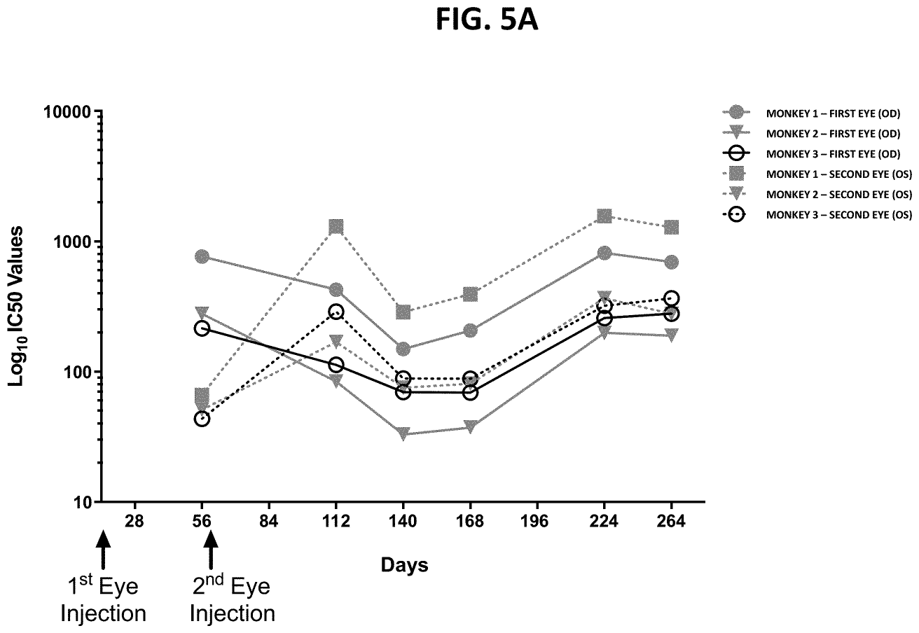

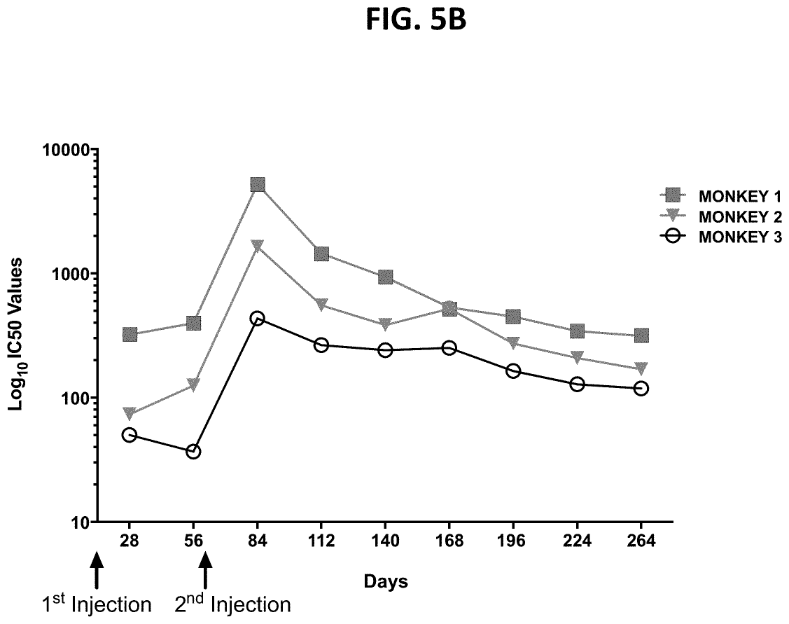

There was an increase in post-IVT AAV2.7m8 nAb response in both the serum and vitreous fluids of the three monkeys administered with AAV2.7m8-aflibercept. See FIGS. 5A and 5B. Greater nAb responses were observed in the later-injected eyes (i.e., OS). Serum nAb levels peaked about 1 month after the second eye injection in each monkey (i.e., ˜Day 84), consistent with recall immune response. Table B below provides a qualitative summary of the data shown in FIGS. 5A and 5B.

TABLE B
Presence of anti-AAV2.7m8 immunoglobulin (IgG) in
serum and vitreous samples at various time points

| | | | | Time Point | | | | | | | | | |
|---|---|---|---|---|---|---|---|---|---|---|---|---|---|
| | | | | | | Month | | Month | | Month | | Month | |
| Sample | | Animal ID | Treatment | Baseline | | 2 | | 4 | | 5 | | 9 | |
| Serum | | Monkey 1 | AAV2.7m8 | − | | + | | + | | + | | + | |
| | | Monkey 2 | AAV2.7m8 | − | | + | | + | | + | | + | |
| | | Monkey 3 | AAV2.7m8 | − | | + | | + | | + | | + | |
| | | Control | Vehicle | − | | − | | − | | − | | − | |
| | | Monkey | | | | | | | | | | | |
| Vitreous | Vitreous | Monkey 1 | AAV2.7m8 | − | − | + | − | + | + | + | + | + | + |
| First | Second | Monkey 2 | AAV2.7m8 | − | − | + | − | + | + | + | + | + | + |
| Eye | Eye | Monkey 3 | AAV2.7m8 | − | − | + | − | + | + | + | + | + | + |
| | | Control | Vehicle | − | − | − | − | − | − | − | − | − | − |
| | | Monkey | | | | | | | | | | | |

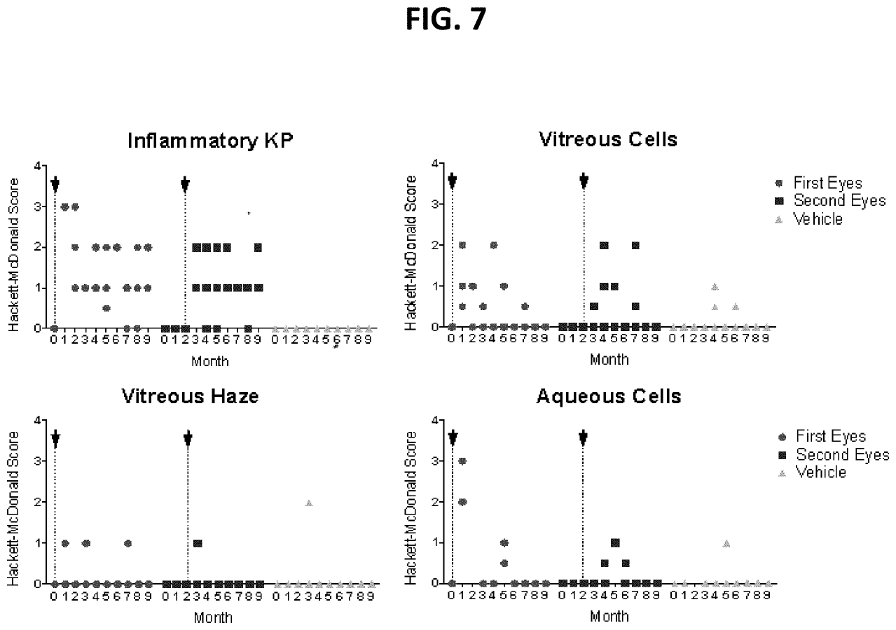

Mild inflammation was observed in both eyes (data not shown), with second injection resulting in earlier onset likely due to recall immune response. Ocular health parameters were scored by the Hackett-McDonald irritation and inflammation scoring system (see, e.g., Hackett, R. B. and McDonald, T. O. “Eye irritation” in Dermatotoxicology, 4th edition, Marzulli, F. N. and Maibach, H. I. editors, Hemisphere Publishing Corp., Washington D.C. (1991)). FIG. 7 shows the levels of inflammatory keratic precipitate (“KP”), vitreous cell infiltrates, vitreous haze, and aqueous cell infiltrates detected, no aqueous flare was detected following IVT injections. (The arrows with dotted lines in FIG. 7 indicate time of injection.) Little to no vitreous haze was detected during slit lamp examination of the eye. Briefly, an aqueous flare is an abnormal appearance of the beam of light as it travels through the anterior chamber. The flare is caused by light reflecting off proteins in the aqueous humor and is typically found when there is inflammation in the anterior chamber. Aqueous and vitreous cell infiltrates were generally transient and self-resolving. The ophthalmic effects summarized in FIG. 7 indicate that bilateral IVT administration AAV2.7m8 was well-tolerated with no serious adverse effects.

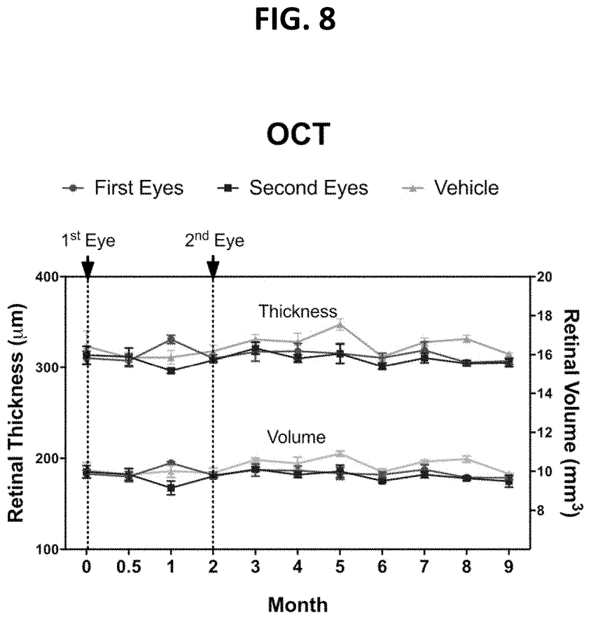

Increase in retinal volume and/or thickening of the retina is indicative of edema, whereas retinal thinning is indicative of loss of retinal cells. Retinal thickness and retinal volume of monkeys that received bilateral IVT administration were as assessed via optical coherence tomography (OCT). As shown in FIG. 8, there was little to no difference in the retinal thickness or retinal volumes of the right eyes (which received the first IVT injections) and left eyes (which received the later IVT injections) in monkeys treated with AAV2.7m8. Moreover, there was little to no difference in the retinal thickness or retinal volumes of monkeys receiving bilateral IVT doses of AAV2.7m8 as compared to monkeys receiving vehicle.

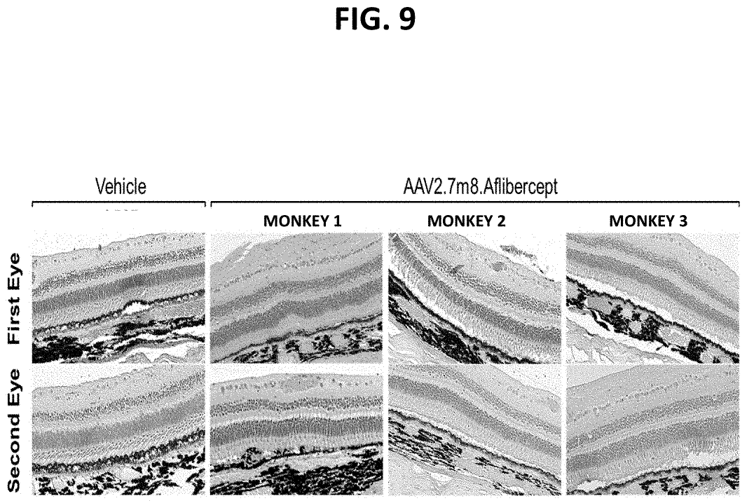

Retinal tissue sections were stained with hematoxyline (which stains nucleic acids) and eosin (which stains proteins) to assess retinal morphology and determine whether cell death occurred and/or whether immune cells infiltrated the retina. FIG. 9 and in Table C below indicate that lesions and perivascular infiltrates were minimal. FIG. 9 also shows that bilateral IVT administration of AAV2.7m8 did not elicit strong immune responses in the monkeys' left (i.e., later-dosed) eyes.

TABLE C
Histopathology Assessment of Retinal Tissues

| Tissue and | Vehicle | | | | | | | |
|---|---|---|---|---|---|---|---|---|
| Finding | Control | | 7m8.CO.Aflibercept | | | | | |
| Animal ID | Monkey | | Monkey 1 | | Monkey 2 | | Monkey 3 | |
| Eye | 1^{st} | 2^{nd} | 1^{st} | 2^{nd} | 1^{st} | 2^{nd} | 1^{st} | 2^{nd} |
| | (OD) | (OS) | (OD) | (OS) | (OD) | (OS) | (OD) | (OS) |
| Grade | NVL | NVL | 1 | 1 | 1 | 1 | 1 | 1 |

Histopathology scoring scale: NVL = no visible lesions; Grade 1 = minimal; Grade 2 = mild; Grade 3 = moderate; Grade 4 = marked; Grade 5 = severe.

The data discussed above demonstrated that development of immunity following IVT administration of AAV2.7m8 capsid in one eye does not completely block transduction following sequential dosing in the contralateral eye. Staggered IVT dosing to both eyes with AAV2.7m8-aflibercept was well-tolerated with minimal perivascular infiltrates and mild inflammation.

Although the present disclosure has been described in some detail by way of illustration and example for purposes of clarity of understanding, the descriptions and examples should not be construed as limiting the scope of the present disclosure. The disclosures of all patent and scientific literature cited herein are expressly incorporated in their entirety by reference.

1: A method of treating an ocular disease or disorder in a subject, comprising:
(i) administering a first unit dose of a pharmaceutical composition to a first eye of the subject via intravitreal (IVT) injection at a first time point, and
(ii) administering a second unit dose of the pharmaceutical composition to a contralateral eye of the subject via IVT injection at a second time point,
wherein the pharmaceutical composition comprises:
(a) a recombinant adeno-associated virus (rAAV) particle comprising a nucleic acid encoding an anti-vascular endothelial growth factor (VEGF) agent, wherein the rAAV particle is capable of infecting a retinal cell following IVT injection and
(b) a pharmaceutically acceptable excipient.

2: The method of claim 1, wherein the method further comprises a step of measuring a level of neutralizing antibodies against the rAAV in a sample from the subject following the first time point and prior to the second time point.

3: The method of claim 1, wherein the method further comprises a step of measuring expression level of the nucleic acid encoding the anti-vascular endothelial growth factor (VEGF) agent in a sample from the subject following the first time point and prior to the second time point.

4: The method claim 1, wherein the time interval between the first time point and the second time point is:
(a) at least about 2 weeks;
(b) at least about 4 weeks or about 1 month;
(c) at least about 6 weeks; or
(d) at least about 8 weeks.

5-7. (canceled)

8: The method of claim 1, wherein the first unit dose and the second unit dose each comprise:
(a) between about 1E9 and about 3E13 vector genomes;
(b) between about 1E10 and about 3E12 vector genomes;
(c) between about 1E11 and 1E13 vector genomes; or
(d) between about 2E11 and 6E12 vector genomes.

9-11. (canceled)

12: The method of claim 1, wherein the second unit dose is higher than the first unit dose.

13: The method of claim 12, wherein the second unit dose is:
(a) at least about 300% of the first unit dose; or
(b) between about 300% and about 1000% of the first unit dose.

14. (canceled)

15: The method of claim 12, wherein:
(a) the first unit dose comprises about 6E10 vector genomes the second unit dose comprises between about 1.8E11 and about 6E11 vector genomes;
(b) the first unit dose comprises about 6E11 vector genomes and the second unit dose comprises between about 1.8E12 and about 6E12 vector genomes;
(c) the first unit dose comprises about 2E11 vector genomes and the second unit dose comprises between about 6E11 and 2E12 about vector genomes; or
(d) the first unit dose comprises about 2E12 vector genomes and the second unit dose comprises between about 6E12 and about 2E13 vector genomes.

16-18. (canceled)

19: The method of claim 1, wherein the volumes of first unit dose and the second unit dose are each no more than about 100 μL or are each no more than about 50 μl.

20. (canceled)

21: A method treating an ocular disease or disorder in a subject, comprising:
administering a unit dose of a pharmaceutical composition to one eye of the subject via intravitreal (IVT) injection,
wherein the pharmaceutical composition comprises:
(a) a recombinant adeno-associated virus (rAAV) particle comprising a nucleic acid encoding an anti-vascular endothelial growth factor (VEGF) agent, wherein the rAAV particle is capable of infecting a retinal cell following IVT injection, and
(b) a pharmaceutically acceptable excipient, and
wherein the subject was administered with a prior unit dose of the pharmaceutical composition to a contralateral eye via IVT injection.

22: The method of claim 21, wherein the method further comprises a step of measuring a level of neutralizing antibodies against the rAAV in a sample from the subject following administration of the prior unit dose to the contralateral eye and prior to the administration of the unit dose to the one eye.

23: The method of claim 21, wherein the method further comprises a step of measuring expression level of the nucleic acid encoding the anti-vascular endothelial growth factor (VEGF) agent in a sample from the subject following administration of the prior unit dose to the contralateral eye and prior to the administration of the unit dose to the one eye.

24: The method of claim 21, wherein the unit dose comprises:
(a) between 1E9 to 3E13 vector genomes;
(b) between 1E10 to 3E12 vector genomes;
(c) between 1E11 and 1E13 vector genomes; or
(d) between 2E11 and 6E12 vector genomes.

25-27. (canceled)

28: The method of claim 21, wherein the prior unit dose comprised:
(a) between 1E9 to 3E13 vector genomes;
(b) between 1E10 to 3E12 vector genomes;
(c) between 1E11 and 1E13 vector genomes; or
(d) between 2E11 and 6E12 vector genomes.

29-31. (canceled)

32: The method of claim 21, wherein the unit dose administered to the one eye is higher than the prior unit dose that was administered to the contralateral eye.

33: The method of claim 32, wherein the unit dose is at least about 300% of the prior unit dose, or between about 300% and about 1000% of the prior unity dose.

34. (canceled)

35: The method of claim 32, wherein:
(a) the prior unit dose comprised about 6E10 vector genomes and the unit dose comprises between about 1.8E11 and about 6E11 vector genomes;
(b) the prior unit dose comprised about 6E11 vector genomes and the unit dose comprises between about 1.8E12 and about 6E12 vector genomes;
(c) the prior unit dose comprised about 2E11 vector genomes and the unit dose comprises between about 6E11 and 2E12 about vector genomes; or
(d) the prior unit dose comprised about 2E12 vector genomes and the unit dose comprises between about 6E12 and about 2E13 vector genomes.

36-38. (canceled)

39: The method of claim 21, wherein the time interval between the administration of the prior unit dose and administration of the unit dose is:
(a) at least about 2 weeks;
(b) least about 4 weeks or about 1 month;
(c) at least about 6 weeks; or
(d) at least about 8 weeks or about two months.

40-42. (canceled)

43: The method of claim 1, wherein the rAAV particle comprises a variant capsid protein that comprises a peptide insertion relative to a corresponding parental AAV capsid protein, wherein the peptide insertion has an amino acid sequence selected from LALGETTRPA (SEQ ID NO: 1); LANETITRPA (SEQ ID NO: 2), LAKAGQANNA (SEQ ID NO: 3), LAKDPKTTNA (SEQ ID NO: 4), KDTDTTR (SEQ ID NO: 5), RAGGSVG (SEQ ID NO: 6), AVDTTKF (SEQ ID NO: 7), STGKVPN (SEQ ID NO: 8), LAKDTDTTRA (SEQ ID NO: 9), LARAGGSVGA (SEQ ID NO: 10), LAAVDTTKFA (SEQ ID NO: 11), and LASTGKVPNA (SEQ ID NO: 12), wherein the insertion site is located between two adjacent amino acids at a position between amino acids corresponding to amino acids 570 and 611 of VP1 of AAV2 or the corresponding position in the capsid protein of another AAV serotype.

44: The method of claim 43, wherein the rAAV particle is an rAAV2 particle that comprises a variant capsid protein that comprises the amino acid sequence LALGETTRPA (SEQ ID NO: 1) inserted between positions 587 and 588 of SEQ ID NO: 13.

45: The method of claim 44, wherein the variant capsid protein comprises the amino acid sequence of SEQ ID NO: 46.

46: The method of claim 1, wherein the rAAV particle comprises a variant capsid protein that comprises a modified sequence, the modified sequence comprising one or more amino acid substitutions within amino acid residues 570-579 relative to a parental AAV capsid protein, wherein the modified sequence comprises HKFKSGD (SEQ ID NO: 37), and wherein the amino acid residue numbering corresponds to an AAV5 VP1 capsid protein.

47: The method of claim 46, wherein the parental AAV capsid protein is:
(a) an AAV5 capsid protein or an AAV5 and AAV2 hybrid capsid protein;
(b) an AAV2.5T capsid protein; or
(c) an AAV2.5T VP1 capsid protein.

48-49. (canceled)

50: The method of claim 46, wherein the modified sequence comprises LAHKFKSGDA (SEQ ID NO: 39).

51: The method of claim 46, wherein the variant AAV capsid protein comprises a capsid sequence having at least 85% homology to the amino acid sequence set forth in SEQ ID NO: 40 or SEQ ID NO: 41.

52: The method of claim 46, wherein the variant AAV capsid protein comprises a capsid sequence set forth in SEQ ID NO: 42 or SEQ ID NO:43.

53: The method of claim 1, wherein the anti-VEGF agent is a bevacizumab, brolucizumab, or ranibizumab.

54: The method of claim 1, wherein the anti-VEGF agent is a polypeptide that comprises an amino acid sequence having at least 80% homology to aflibercept.

55: The method of claim 54, wherein the anti-VEGF agent is aflibercept.

56: The method of claim 1, wherein the retinal cell is a photoreceptor, a retinal ganglion cell, a Müller cell, a bipolar cell, an amacrine cell, a horizontal cell, or a retinal pigmented epithelium cell.

57: The method of claim 1, wherein the ocular disease or disorder is choroidal neovascularization, wet age-related macular degeneration (wAMD), macular edema following retinal vein occlusion, diabetic macular edema (DME), or diabetic retinopathy associated with DME.

58. (canceled)

59: The method of claim 1, wherein the subject is a human.

60: The method of claim 1, wherein the subject is responsive to administration of an anti-VEGF agent, wherein the anti-VEGF agent is a polypeptide.

61: The method of claim 60, wherein the anti-VEGF agent is aflibercept.

62: The method of claim 1, wherein the subject received prior treatment for the ocular disease or disorder with an anti-VEGF agent.

63: The method of claim 62, wherein the anti-VEGF agent was aflibercept.
